(19) United States
(12) Patent Application Publication
(10) Pub. No.: US 2009/0075863 A1
(43) Pub. Date: Mar. 19, 2009

(54) Periosteal Tissue Grafts

(75) Inventors: Shawn W. O'Driscoll, Rochester, MN (US); Gregory G. Reinholz, Rochester, MN (US); James S. Fitzsimmons, Rochester, MN (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 12/171,117

(22) Filed: Jul. 10, 2008

Related U.S. Application Data
(60) Provisional application No. 60/949,231, filed on Jul. 11, 2007.
(60) Provisional application No. 60/959,081, filed on Jul. 10, 2007.

(51) Int. Cl. A61K 38/30; A61K 38/18; A61P 43/00

(52) U.S. Cl. 514/3; 514/12

(57) ABSTRACT

This document relates to methods and materials involved in making and using periosteal tissue grafts in vivo. For example, materials and methods for obtaining grafts comprising periosteal tissue are provided.

### CROSS-REFERENCE TO RELATED APPLICATIONS

This application claims the benefit of U.S. Provisional Application Ser. No. 60/949,231, filed Jul. 11, 2007 and U.S. Provisional Application Ser. No. 60/959,081, filed Jul. 10, 2007. The disclosures of the prior applications are considered part of (and are incorporated by reference in) the disclosure of this application.

### STATEMENT AS TO FEDERALLY SPONSORED RESEARCH

This invention was made with government support under AR052115 awarded by National Institutes of Health. The government has certain rights in the invention.

### BACKGROUND

1. Technical Field

This document relates to methods and materials involved in making and using periosteal tissue grafts in vivo.

2. Background Information

Articular cartilage is the remarkably durable tissue that covers the articulating bone surfaces in our joints and permits pain-free movement by greatly reducing friction between bones and distributing stress. Unfortunately, when cartilage is damaged due to injury or disease, it has a limited capacity to heal and can lead to premature arthritis. Approximately one out of every six Americans are affected by arthritis and that number is predicted to increase as our population ages. Adult articular cartilage contains no blood supply, neural network, or lymphatic drainage. As a result, partial thickness defects that do not reach the subchondral bone stimulate only a transient induction of chondrocyte replication and matrix production in the area adjacent to the wound. An inflammatory response that is typical of wound healing in other tissues is only observed in full thickness defects that penetrate the subchondral bone. In this case, the defect is exposed to bone marrow, and cells are recruited to fill the defect with new tissue. The extent to which the new tissue resembles articular cartilage depends on the age and species of the host as well as the size and location of the defect. However, complete restoration of the hyaline articular cartilage and the subchondral bone to a normal status is rarely seen.

### SUMMARY

This document relates to methods and materials involved in making and using periosteal tissue grafts in vivo. For example, this document provides methods and materials for creating rejuvenated periosteal tissue in vivo. As described herein, in vivo periosteal tissue can be treated with one or more growth factors (e.g., TGF-β, IGF-1, or both TGF-β and IGF-1) via one or more (e.g., between one and ten) injections, which can be evenly spaced within the tissue to be harvested. In some case, periosteal tissue can be treated in vivo with one or more growth factors on a single day or daily for up to 14 days (e.g., 2, 3, 4, 5, 6, 7, 8, 9, 10, 11, 12, 13, or 14 days). One to 20 days (e.g., 1, 2, 3, 4, 5, 6, 7, 8, 9, 10, 11, 12, 13, 14, 15, 16, 17, 18, 19, or 20 days) after the initial injection, the in vivo-treated periosteal tissue can be harvested and used as a rejuvenated periosteal tissue graft. Such grafts can be used to regenerate cartilage, bone, or cartilage and bone.

In general, one aspect of this document features a method for obtaining a graft comprising periosteal tissue. The method comprises, or consists essentially of, (a) administering a composition to periosteal tissue of a mammal to form in vivo-treated periosteal tissue, wherein the composition comprises a growth factor, and (b) harvesting the in vivo-treated periosteal tissue from the mammal, thereby obtaining the graft. The mammal can be a human. The periosteal tissue can be in an arm of the mammal. The periosteal tissue can be in a leg of the mammal. The growth factor can be a transforming growth factor or an insulin-like growth factor. The transforming growth factor can be a transforming growth factor-beta polypeptide. The insulin-like growth factor can be a insulin-like growth factor-1 polypeptide. The insulin-like growth factor can be a insulin-like growth factor-2 polypeptide. The composition can comprise a transforming growth factor and an insulin-like growth factor. The administration can be a subperiosteal injection.

In another aspect, this document features a method for regenerating cartilage or bone in a mammal. The method comprises, or consists essentially of, inserting a periosteal tissue graft into the mammal, wherein the periosteal tissue graft was harvested from the mammal after being treated in vivo with a composition comprising a growth factor. The mammal can be a human. The periosteal tissue could have been harvested from an arm of the mammal. The periosteal tissue could have been harvested from a leg of the mammal. The growth factor can be a transforming growth factor or an insulin-like growth factor. The transforming growth factor can be a transforming growth factor-beta polypeptide. The insulin-like growth factor can be a insulin-like growth factor-1 polypeptide. The insulin-like growth factor can be a insulin-like growth factor-2 polypeptide. The composition can comprise a transforming growth factor and an insulin-like growth factor. The periosteal tissue graft could have been treated via a subperiosteal injection of the composition.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1: IGFBP-4 Protease Assay. Conditioned medium was collected from periosteal explants cultured with or without 10 ng/mL TGF-β1 for the first 48 hours. A) Western ligand blot. B) Densitometry analysis of western ligand blot.

FIG. 2: PAPP-A protein levels in conditioned medium from periosteal explants cultured with or without 10 ng/mL TGF-β1 for the first 48 hours.

FIG. 3: A) Cartilage Yield. B) Total Cartilage (% Cartilage×Wet Weight).

FIG. 4: Proliferation results on days 3 and 14 normalized to total DNA.

FIG. 5: Tissue outgrowth from periosteum/tantalum composites after six weeks of culture. (A) Composite with cambium layer facing down to the tantalum. (B) Composite with cambium layer facing up away from the tantalum.

FIG. 6: Histological examination of periosteum/tantalum composites. Examples of composites after six weeks of culture in chondrogenic medium. The composites were embedded and sectioned using the Exakt™ System and stained using safranin O/fast green (shown in grayscale). (A) Composite with cambium layer facing down to the tantalum (5×). (B) Higher magnification of inset from A (10×). (C) Composite with cambium layer facing up from the tantalum (5×). (D) Higher magnification of inset from C (10×). Open arrow=tantalum. Closed arrow=tissue outgrowth.

FIG. 7: Mechanical properties analysis of periosteum/tantalum composites compared to osteochondral plugs. Periosteal/tantalum composites were cultured for six weeks with 10 ng/mL TGF-β supplementation for the first two days. The composites and normal osteochondral plugs were then subjected to indentation analysis. The composites had mechanical properties comparable to the osteochondral plug controls as can be seen by the similarly shaped curves.

FIG. 8: (Left) Cross-section of periosteum on bone (H&E). (Right) Thickness of the cambium layer is measured in micrometers. To determine the cell density, all of the nuclei and nuclear fragments within a 40×150 μm rectangle are counted and expressed as cells/μm². The total cell count can be defined as the product of the cambium layer thickness, the width of the sampling rectangle (150 μm), and the cell density.

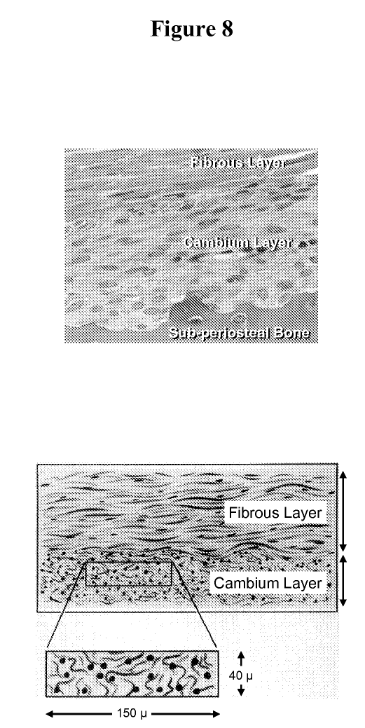

FIG. 9: Example of computerized histomorphometry of a periosteal explant stained with safranin O/fast green (shown in grayscale). % cartilage is determined based on the uptake of safranin O stain (red; shown in grayscale).

FIG. 10: Autologous periosteal transplantation surgical procedure. t=tibia, d=defect, p=periosteal graft, s=suture, and cl=cambium layer.

FIG. 11: Schematic drawing of the medial proximal rabbit tibia showing the region bounded by the patellar ligament, growth plate, and medial collateral ligament that was used for harvesting 3.5×3.5 mm² periosteal explants (grid). The four percutaneous injections were targeted in these regions (circles).

FIG. 12: Cambium cellularity (A and D), maximum cambium thickness (B and E) and cambium cell density (C and F) of periosteum from 12-month old rabbit tibia measured 1, 3, 5, or 7 days post injection of vehicle, TGF-β1 (20, or 200 ng) (A-C), or TGF-β1 (200 ng) plus IGF-1 (2000 ng) (D-F). Lowercase letters indicate the results of post-hoc testing using least squares means differences Student's t-test (p<0.05). Columns with letters in common are not statistically different from one another.

FIG. 13: Typical (representative of the means) H & E stained histological specimens of periosteum on-bone, from 6 and 12 month-old rabbits 7 days after injection with 200 ng TGF-β1 or 200 ng TGF-β1 plus IGF-1. The lines and arrows indicate the cambium.

FIG. 14: Typical (representative of the means) H & E stained histological specimens of periosteum on-bone, from 12 month-old rabbits 1, 3, 5, or 7 days after injection with 200 ng TGF-β1 or 200 ng TGF-β1 plus IGF-1. The lines and arrows indicate the cambium.

FIG. 15: Cartilage Yield (A and D), explant wet weight (B and E), and total cartilage index (C and F) of periosteum from 12-month old rabbit tibia measured 1, 3, 5, or 7 days post injection of vehicle, TGF-β1 (20, or 200 ng) (A-C), or TGF-β1 (200 ng) plus IGF-1 (2000 ng) (D-F). Lowercase letters indicate the results of post-hoc testing using least squares means differences Student's t-test (p<0.05). Columns with letters in common are not statistically different from one another.

FIG. 16: Cartilage Yield (A and D), explant wet weight (B and E), and total cartilage index (C and F) of periosteum from 2 year-old rabbit tibia measured 1, 3, 5, or 7 days post injection of vehicle, TGF-β1 (200 ng) (A-C), or TGF-β1 (200 ng) plus IGF-1 (2000 ng) (D-F). Lowercase letters indicate the results of post-hoc testing using least squares means differences Student's t-test (p<0.05). Columns with letters in common are not statistically different from one another.

FIG. 17: Correlation between the total cartilage index and cambium cellularity from 12-month old rabbit periosteum injected with vehicle or TGF-β1 (200 ng) and harvested 1, 3, 5, or 7 days post injection.

FIG. 18: Typical (representative of the means) Safranin O/fast green stained histological specimens of cultured periosteum harvested from 6, 12 and 24-month old rabbits 7 days after injection of vehicle, TGF-β1 (200 ng), or TGF-β1 (200 ng) plus IGF-1 (2000 ng).

FIG. 19: Gross images (A & C) and safranin O/fast green (shown in grayscale) stained histological sections (B & D) of regenerated tissue 6 weeks after implantation of TGF-β1-injected (A & B) and non-injected (C & D) periosteal grafts.

FIG. 20: Analyses of 6-week post-op regenerated osteochondral tissue. A) Bone histological score. B) Cartilage histological score. C) Total histological score. D) Equilibrium modulus. Lowercase letters indicate the results of post-hoc testing using Student's test (p<0.05). Columns with letters in common are not statistically different from one another.

### DETAILED DESCRIPTION

This document provides methods and materials related to making and using periosteal tissue grafts in vivo. In some cases, factors such as growth hormone or FGF-2 can be used individually, together, or in combination with IGF-1 and/or TGF-β as described herein. Compositions containing one or more factors can be formulated for slow-release to prolong bioavailability. In some cases, the methods and materials provided herein can be used to engineer bone.

Experiments can be performed to determine whether subperiosteal injection of specific growth factors can be used as a pretreatment to enhance the chondrogenic potential of autologous periosteal grafts for cartilage repair. The potential for subperiosteal injection of growth factors to (1) expand the number of chondrogenic precursor cells in periosteum in vivo, (2) enhance the in vitro chondrogenic potential of periosteum, and (3) improve the potential of periosteum to repair articular cartilage defects can be examined. In particular, experiments can be performed to determine (1) the effects of subperiosteal growth factor injection on cambium layer thickness and periosteal cell proliferation and differentiation in situ over time, (2) the effects of subperiosteal growth factor injection on subsequent periosteal chondrogenesis in vitro, and (3) the effects of subperiosteal growth factor injection on biological resurfacing of articular cartilage by periosteal transplantation. When cells incorporate naturally into the surrounding matrix of the periosteum during “activated” proliferation, the need for in vitro cell seeding can be eliminated. For example, the engineered tissue can be prepared for transplantation in vivo.

The growth factors TGF-β1 and IGF-1 can be used for these experiments. TGF-β1 alone, IGF-1 alone, or a combination of TGF-β1 and IGF-1 can be injected into the subperiosteal region of the medial side of the proximal tibia of 6 and 12-month old New Zealand white rabbits. At various time points, the periosteum can be harvested and analyzed for cambium layer thickness, proliferation, and differentiation. The optimum concentration of growth factor and timing of periosteal harvest can be determined in order to maximize the pool of mesenchymal stem cells prior to in vivo chondrocyte differentiation for subsequent chondrogenesis after transplantation into an articular cartilage defect.

128 New Zealand white rabbits (64 six month-old and 64 twelve month-old) can be administered 10 μL subperiosteal injections containing 20 or 200 ng of TGF-β1 or vehicle (4 mM HCl, 1 mg/mL BSA), or 200 ng or 2 μg of IGF-1 or vehicle (10 mM acetic acid, 0.1% BSA). One limb of each rabbit can receive growth factor, while the other limb can be injected with vehicle. The injections can be made into the subperiosteal region of the medial side of the proximal tibia (Tables 1 and 2). Four injections can be made in each limb spaced apart in a manner to center them over four 2×3 mm areas normally used to harvest periosteal explants. Injecting at four sites can allow four samples to be harvested from each limb. The average values of these four harvest sites can be used for each animal. Thus, multiple measures can be obtained from each animal in order to maximize the data obtained from each rabbit. The multiple measures approach can help to eliminate errors that can occur due to miss-injection, for example. In addition, multiple site injections can provide a wider distribution of the growth factors, which can be important for harvesting the larger explants for defect repair. After 1, 3, 5, or 7 days, the rabbits can be sacrificed, and the periosteum with the underlying tibia bone can be harvested from the injection sites. The samples can be fixed and embedded “on edge” in paraffin to obtain cross-sections of the tissue. H & E staining can be performed, and the cambium layer thickness and total cell count can be measured as described elsewhere (Gallay et al., J. Orthop. Res., 12:515-525 (1994); O'Driscoll et al., J. Orthop. Res., 19:95-103 (2001)). To detect and localize proliferating cells, immunohistochemistry for proliferative cell nuclear antigen (PCNA) can be performed on sections as described elsewhere (Fukumoto et al., Osteoarthritis Cartilage, 11:55-64 (2003)). In order to determine the onset new cartilage formation, sections can be stained with Safranin O/fast green (O'Driscoll et al., Tissue Eng., 5:13-23 (1999)). The presence of new bone can be assessed using Masson's trichrome stain (Joyce et al., J. Cell. Biol., 110:2195-2207 (1990)).

TABLE 1
Summaries for experiments involving the subperiosteal injection of different
doses of TGF-β1 and the vehicle control (Ctrl.) for histological/histochemical evaluation
1, 3, 5, and 7 days after injection

| | 6 Month Rabbits | | | | 12 Month Rabbits | | | |
|---|---|---|---|---|---|---|---|---|
| Days | 20 ng/mL | | 200 ng/mL | | 20 ng/mL | | 200 ng/mL | |
| Pre-Treatment | TGF-β1 | Ctrl. | TGF-β1 | Ctrl. | TGF-β1 | Ctrl. | TGF-β1 | Ctrl. |
| 1 | Rb 1-4 | Rb 1-4 | Rb 17-20 | Rb 17-20 | Rb 33-36 | Rb 33-36 | Rb 49-52 | Rb 49-52 |
| 3 | Rb 5-8 | Rb 5-8 | Rb 21-24 | Rb 21-24 | Rb 36-40 | Rb 37-40 | Rb 53-56 | Rb 53-56 |
| 5 | Rb 9-12 | Rb 9-12 | Rb 25-28 | Rb 25-28 | Rb 41-44 | Rb 41-44 | Rb 57-60 | Rb 57-60 |
| 7 | Rb 13-16 | Rb 13-16 | Rb 29-32 | Rb 29-32 | Rb 45-48 | Rb 45-48 | Rb 61-64 | Rb 61-64 |

Numbers in Table 1 and subsequent tables indicate rabbit usage (Rb = “Rabbit”).

TABLE 2
Summaries for experiments involving the subperiosteal injection of different
doses of IGF-1 and the vehicle control (Ctrl.) for histological/histochemical evaluation 1,
3, 5, and 7 days after injection*

| | 6 Month Rabbits | | | | 12 Month Rabbits | | | |
|---|---|---|---|---|---|---|---|---|
| Days | 200 ng/mL | | 2 μg/mL | | 200 ng/mL | | 2 μg/mL | |
| Pre-Treatment | IGF-1 | Ctrl. | IGF-1 | Ctrl. | IGF-1 | Ctrl. | IGF-1 | Ctrl. |
| 1 | Rb 1-4 | Rb 1-4 | Rb 17-20 | Rb 17-20 | Rb 33-36 | Rb 33-36 | Rb 49-52 | Rb 49-52 |
| 3 | Rb 5-8 | Rb 5-8 | Rb 21-24 | Rb 21-24 | Rb 36-40 | Rb 37-40 | Rb 53-56 | Rb 53-56 |
| 5 | Rb 9-12 | Rb 9-12 | Rb 25-28 | Rb 25-28 | Rb 41-44 | Rb 41-44 | Rb 57-60 | Rb 57-60 |
| 7 | Rb 13-16 | Rb 13-16 | Rb 29-32 | Rb 29-32 | Rb 45-48 | Rb 45-48 | Rb 61-64 | Rb 61-64 |

*Rb = “Rabbit”

The outcomes of primary interest can be normalized to cell number (cells/mm), proliferation index (i.e., PCNA cell counting), and cartilage formation. Separate analyses can be conducted for each outcome. In order to evaluate the effects of treatment (TGF-β1 or IGF-1 vs. control), dosage (20 ng/mL vs. 200 ng/mL TGF-β or 200 ng/mL vs. 2 μg/mL IGF-1) and time (1, 3, 5, or 7 days), a 3-factor analysis of variance with repeated measures on one factor (treatment) can be performed. When no significant interactions are observed, main effects can be estimated and reported. When the main effect of time is found to be significant, further analysis can be performed using an appropriate multiple comparisons procedure or contrast statements. When significant interactions are found, then separate lower-order models can be utilized. For each outcome, separate analyses can be performed for the 6 month-old rabbits and the 12 month-old rabbits. All statistical tests can be two-sided, and p-values less than 0.05 can be considered significant.

Sixty-four rabbits can be assigned to the various treatment combinations as indicated in Tables 1 and 2. As noted herein, separate analyses can be conducted for the 6 month-old and 12 month-old rabbits. Data on untreated periosteum still on the underlying bone in 6 month-old rabbits (O'Driscoll et al., J. Orthop. Res., 19:95-103 (2001)) indicate an average normalized cell number of 80 cells/mm with a standard deviation of 27. When similar data are observed, there can be 80% power to detect a difference of 14 cells/mm between TGF-β1 or IGF-1 and control, a difference of 30 cells/mm between doses (20 ng/mL and 200 ng/mL TGF-β1 or 200 ng/mL and 2 μg/mL IGF-1), and a difference of 44 cells/mm between any two of the four sacrifice times (1, 3, 5, and 7 days) for TGF-β1 or IGF-1.

For PCNA cell counting in the TGF-β1-treated groups, data presented elsewhere show a mean proliferative index of 61±7 in vitro with TGF-β treatment (Fukumoto et al., Osteoarthritis Cartilage, 11:55-64 (2003)). When similar results are achieved, there can be 80% power to detect a difference of 3.6 between TGF-β1 and control, a difference of 7.4 between a 20 ng/mL dose and 200 ng/mL dose, and a difference of 11.4 between any two of the four sacrifice times (1, 3, 5, and 7 days). For PCNA cell counting in the IGF-1 treated groups, data presented elsewhere show a mean proliferative index of 84±3 in vitro with IGF-1 treatment (Fukumoto et al., Osteoarthritis Cartilage, 11:55-64 (2003)). When similar results are achieved, there can be 80% power to detect a difference of 1.6 between IGF-1 and control, a difference of 3.2 between a 200 ng/mL dose and a 2 μg/mL dose, and a difference of 4.9 between any two of the four sacrifice times (1, 3, 5, and 7 days).

Subperiosteal injections of TGF-β1 and IGF-1 combined in vivo can “activate” the periosteum of skeletally mature rabbits to proliferate and form more cartilage after subsequent harvest. 32 New Zealand white rabbits (16 six-month and 16 twelve month-old) can receive 10 μL subperiosteal injections containing TGF-β1 plus IGF-1 or vehicle (5 mM acetic acid, 2 mM HCL, 0.1% BSA). The concentration of TGF-β1 and IGF-1 used in this combined treatment can be determined by the effective doses of each alone. One limb of each rabbit can receive TGF-β1 and IGF-1, while the other limb can be injected with vehicle (Table 3). The injections can be made into the subperiosteal region of the medial side of the proximal tibia. Four injections can be made in each limb spaced apart in a manner to center them over four 3.5×3.5 mm areas normally used to harvest periosteal explants. After 1, 3, 5, or 7 days, the rabbits can be sacrificed, and the periosteum with the underlying tibia bone can be harvested from the injection sites. The samples can be fixed and embedded “on edge” in paraffin to obtain cross-sections of the tissue. H & E staining can be performed, and the cambium layer thickness and total cell count can be measured as described elsewhere (Gallay et al., J. Orthop. Res., 12:515-525 (1994) and O'Driscoll et al., J. Orthop. Res., 19:95-103 (2001)). To detect and localize proliferating cells, immunohistochemistry for proliferative cell nuclear antigen (PCNA) can be performed on sections as described elsewhere (Fukumoto et al., Osteoarthritis Cartilage, 11:55-64 (2003)). In order to determine the onset new cartilage formation, sections can be stained with Safranin O/fast green (O'Driscoll et al., Tissue Eng., 5:13-23 (1999)). The presence of new bone can be assessed using Masson's trichrome stain (Joyce et al., J. Cell. Biol., 110:2195-2207 (1990)).

TABLE 3
Summaries for experiments involving the subperiosteal injection of
different doses of TGF-β1 and IGF-1 and the vehicle control
(Ctrl.) for histological/histochemical evaluation 1, 3, 5, and 7
days after injection.

| | 6 m Rabbits | | 12 m Rabbits | |
|---|---|---|---|---|
| Days | TGF-β1 + | | TGF-β1 + | |
| Pre-Treatment | IGF-1 | Ctrl. | IGF-1 | Ctrl. |
| 1 | Rb 1-4 | Rb 1-4 | Rb 17-20 | Rb 17-20 |
| 3 | Rb 5-8 | Rb 5-8 | Rb 21-24 | Rb 21-24 |
| 5 | Rb 9-12 | Rb 9-12 | Rb 25-28 | Rb 25-28 |
| 7 | Rb 13-16 | Rb 13-16 | Rb 29-32 | Rb 29-32 |

(Rb = “Rabbit”).

The outcomes of primary interest are cambium thickness (μm), cambium cellularity (cells/mm) and cell density (cells/cm²), and cartilage formation. In some cases, the outcomes of interest can be normalized to cell number (cells/mm), proliferative index (i.e., PCNA cell counting), and cartilage formation. Separate analyses can be conducted for each outcome. In order to evaluate the effects of treatment (TGF-β1+IGF-1 vs. control) and time (1, 3, 5, 7 days), a 2-factor analysis of variance with repeated measures on one factor (treatment) can be performed. When no significant interaction is observed, main effects can be estimated and reported. When the main effect of time is found to be significant, further analysis can be performed using an appropriate multiple comparisons procedure or contrast statements. When no significant interaction is found, then separate one-factor models can be utilized. For each outcome, separate analyses can be performed for the 6 month-old rabbits and the 12 month-old rabbits. All statistical tests are two-sided, and p-values less than 0.05 can be considered significant.

Thirty-two rabbits can be assigned to the various treatment combinations as indicated in Table 3. As noted herein, separate analyses can be conducted for the 6 month-old and 12 month-old rabbits. Data on untreated periosteum still on the underlying bone in 6 month-old rabbits (O'Driscoll et al., J. Orthop. Res., 19:95-103 (2001)) indicate an average normalized cell number of 80 cells/mm with a standard deviation of 27. When similar data are observed in this experiment, there can be 80% power to detect a difference of 21 cells/mm between TGF-β1+IGF-1 and control and a difference of 79 cells/mm between any two of the four sacrifice times (1, 3, 5, and 7 days).

For PCNA cell counting, data presented elsewhere show a mean proliferative index of 81±3 with in vitro TGF-β1+IGF-1 treatment (Fukumoto et al., Osteoarthritis Cartilage, 11:55-64 (2003)). When similar results are achieved, there can be 80% power to detect a difference of 2.3 between TGF-β1+IGF-1 and control and a difference of 8.9 between any two of the four sacrifice times (1, 3, 5, and 7 days).

Using the same pretreatment regimen described above for TGF-β1 and IGF-1 alone and in combination, the effect of subperiosteal growth factor injection on subsequent periosteal chondrogenesis can be examined. TGF-β1 and IGF-1 alone and in combination can be injected into the subperiosteal region of the medial side of the proximal tibia of 6-month, 12-month, and 24-month old New Zealand white rabbits. After various times, the periosteum can be harvested and cultured in agarose suspension without additional growth factor supplement. An effective concentration of growth factor and timing of periosteal harvest can be determined in order to maximize the pool of mesenchymal stem cells prior to in vivo chondrocyte or osteoblast differentiation for subsequent chondrogenesis after transplantation into an articular cartilage defect. A pretreatment regimen can be chosen that can have the best chance of success in the cartilage defect experiments.

Subperiosteal injection of TGF-β1 and IGF-1 alone or in combination can “activate” the periosteum of skeletally mature rabbits to proliferate and increase the number of chondrocyte precursors in the cambium layer and enhance subsequent chondrogenesis. 160 New Zealand white rabbits (80 six month-old and 80 twelve month-old) can receive 10 μL subperiosteal injections containing 20 or 200 ng TGF-β1 or vehicle (4 mM HCl, 1 mg/mL BSA; Table 1), 200 ng or 2 μg of IGF-1 or vehicle (10 mM acetic acid, 0.1% BSA; Table 2) or TGF-β1 plus IGF-1 or vehicle (5 mM acetic acid, 2 mM HCL, 0.1% BSA; Table 3). The concentration of TGF-β1 and IGF-1 used in the combined treatment can be determined by the effective dose of each alone. One limb of each rabbit can receive growth factor while the other limb can be injected with vehicle. The injections can be made into the subperiosteal region of the medial side of the proximal tibia. Four injections can be made in each limb spaced apart in a manner to center them over four 2×3 mm areas normally used to harvest periosteal explants. After 1, 3, 5, or 7 days, the rabbits can be sacrificed and the periosteum can be harvested from the injection sites by sharp elevation. The explants can then be cultured with DMEM 10% FBS in agarose suspension without additional growth factors. The explants can then be harvested, embedded in paraffin, sectioned and stained with Safranin O/fast green (O'Driscoll et al., Tissue Eng., 5:13-23 (1999)). The cartilage formation in the explants can be determined using computerized histomorphometry techniques (O'Driscoll et al., Tissue Eng., 5:13-23 (1999)). The formation of mineralized matrix can be detected using Von Kossa stain.

Cartilage formation can be the main outcome analyzed. As described herein, the factors under investigation can be growth factor (TGF-β1 vs. control, IGF-1 vs. control, and TGF-β1+IGF-1 vs. control), dosage (20 ng/mL vs. 200 ng/mL of TGF-β1, and 200 ng/mL vs 2 μg/mL of IGF-1) and time (1, 3, 5, 7 days). The analysis plan for these experiments can be identical to those described above. For example, a 3-factor analysis of variance with repeated measures on one factor (treatment) can be performed. In the absence of significant interactions, main effects can be estimated and reported. When the main effect of time is found to be significant, further analysis can be performed using an appropriate multiple comparisons procedure or contrast statements. When significant interactions are found, then separate lower-order models can be utilized. For each outcome, separate analyses can be performed for the 6 month-old rabbits and the 12 month-old rabbits. All statistical tests can be two-sided, and p-values less than 0.05 can be considered significant.

Sixty-four rabbits can be assigned to the various treatment combinations as indicated in Table 1. As noted above, separate analyses can be conducted for the 6 month-old and 12 month-old rabbits. The average cartilage yield can be about 25%±21%. When similar results are obtained, there can be 80% power to detect a difference of 11 percentage points between treatment (TGF-β1, IGF-1, or combination) and control, a difference of 22 percentage points between dosage levels, and a difference of 34 percentage points between any two of the four sacrifice times (1, 3, 5, and 7 days).

Subperiosteal injection of TGF-β1 and IGF-1 alone or in combination can “activate” the periosteum of skeletally mature rabbits to proliferate and increase the number of chondrocyte precursors in the cambium layer and enhance subsequent biological resurfacing of articular cartilage by periosteal transplantation. 140 New Zealand white rabbits (70 six month-old and 70 twelve month-old) can receive subperiosteal injections of growth factors or vehicle control (Table 4). The injections can be made into the subperiosteal region of the medial side of the proximal tibia. The “activated” and control periosteal grafts can then be harvested and used in autologous periosteal transplantation as described elsewhere (O'Driscoll et al., J. Bone Joint Surg., 68A: 1017-1035 (1986) and O'Driscoll et al., J. Bone Joint Surg., 70A:595-606 (1988)). These grafts can be sutured into the base of five by ten-millimeter full-thickness defects in the patellar groove of experimental rabbits with the cambium layer facing towards the joint. Control rabbits can each receive a periosteal graft that has not been pretreated by subperiosteal injection. After surgery, the rabbits can be allowed unrestricted motion for 6 weeks, 6 months, or 1 year. At the time of sacrifice, operated knees can be harvested and split in two along the patellar groove. One randomly chose half can be analyzed by a scoring system described elsewhere (O'Driscoll et al., J. Bone Joint Surg., 68A:1017-1035 (1986) and O'Driscoll et al., J. Bone Joint Surg., 70A:595-606 (1988)). From the remaining half, a core biopsy can be taken from the center of the defect repair region using a 3.5 mm dermal punch and can undergo mechanical testing. After mechanical testing is complete, this biopsy along with the repair tissue that originally surrounded it can be assayed for type II collagen formation.

TABLE 4
Summaries for experiments involving the subperiosteal injection of growth
factors and the vehicle control (Ctrl.) prior to autologous periosteal
transplantation repair of 5 × 10 mm patellar groove defects.
Histological/histochemical evaluation of the repair will be done
at 6 weeks, 6 months, and 1 year post-op.

| Post-OP | 6 month | | 12 month | |
|---|---|---|---|---|
| Evaluation | Repair | Ctrl. | Repair | Ctrl. |
| 6 weeks | Rb 1-11 | Rb 12-22 | Rb 71-81 | Rb 82-92 |
| 6 months | Rb 23-34 | Rb 35-46 | Rb 93-104 | Rb 105-116 |
| 1 year | Rb 47-58 | Rb 59-70 | Rb 117-128 | Rb 129-140 |

(Rb = “Rabbit”).

The analysis outcomes can include quantitative collagen typing and cartilage scoring. Separate analyses can be conducted for each of these two outcomes. The experimental factors can be treatment (periosteal graft repair of defect vs. defect only) and sacrifice time (6 weeks, 6 months, and 1 year). See Table 4. For each outcome, separate analyses can be performed for the 6 month-old rabbits and the 12 month-old rabbits. The data can be analyzed using 2-factor analysis of variance. When no significant interaction is observed, main effects can be estimated and reported. When the main effect of sacrifice time is found to be significant, further analysis can be performed using an appropriate multiple comparisons procedure or contrast statements. However, if a significant interaction is found, then separate one-factor ANOVA models can be used. All statistical tests can be two-sided, and p-values less than 0.05 can be considered significant.

One hundred and forty rabbits can be assigned to the various treatment combinations as indicated in Table 4. This can include additional animals to account for attrition due to the long experimental times. For example, this can allow for a potential 10% attrition rate in the 6-week group, and a potential 20% attrition rate in the 6 month and 1 year groups. As noted above, separate analyses can be conducted for the 6 month-old and 12 month-old rabbits. Data presented elsewhere (O'Driscoll et al., J. Bone Joint Surg., 70A:595-606 (1988)) suggest an expected average type TI collagen content of 15%±5% for the control animals. When similar findings are obtained, there can be 80% power to detect a difference of 3.7 percentage points between treatment and control, and a difference of 4.7 percentage points between any two of the three sacrifice times. For cartilage scoring, data presented elsewhere (O'Driscoll et al., J. Bone Joint Surg., 70A:595-606 (1988)) indicate that the mean score can be about 12.2±2.6 points. When similar results are observed, the specified sample size can provide 80% power to detect a difference in means of 2.0 points between treatment and control, and a difference of 2.4 points between any two of the three sacrifice times.

The following can be used for assessing the methods and materials provided herein in rabbits or can be adapted for use in other mammal such as humans.

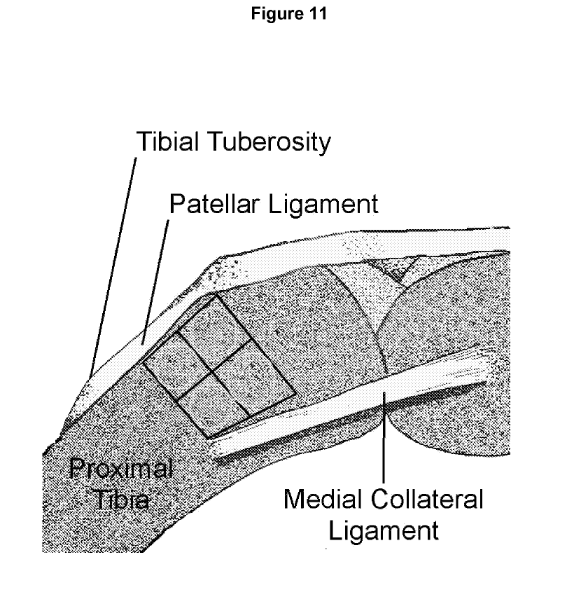

Subperiosteal injection: Percutaneous, subperiosteal injections can be made according to the method described elsewhere (Critchlow et al., J. Cell Sci., 107:499-516 (1994)). Using a Hamilton syringe with a 26-gauge needle, microinjections can be directed into 4 sites in each medial proximal tibia which correspond to the four 3.5×3.5 mm regions that are normally used for periosteal harvesting (FIG. 11). Once the first injection is made, the needle can be left in place while the remaining three injections are made so as to allow accurate positioning of the injections with respect to each other. One limb can receive the growth factor injections, and the contralateral limb can receive injections of vehicle only. After 1, 3, 5, or 7 days after the injections, rabbits can be euthanized by lethal injection using an overdose of sodium pentobarbital (26% v/v), which can be administered through the marginal vein of one of the ears. The animal's death can be verified by checking for a pulse and a blink reflex to tactile stimulus. Osteoperiosteal or periosteal tissue specimens (e.g., explants) can then be collected using a bone saw or sharp sub periosteal dissection respectively and analyzed or cultured.

Cambium Layer Morphology Analysis: Periosteal morphology can be measured in each of the injection regions with the periosteum intact on the bone. A bone saw can be used to harvest the medial proximal tibiae region containing the injection sites. These samples can then be decalcified. After decalcification, the injection sites can be separated and individually paraffin embedded. Each of these samples can be oriented “on edge” during the embedding process to yield cross-sections of the cambium layer and underlying bone. Samples can be sectioned and stained with H&E. The cambium layer thickness and total cell count can be measured at three randomly selected 40 μm rectangles for each of the four injection sites per tibiae sample.

Proliferation Assessment and Localization: To detect and localize the cells undergoing proliferation, immunohistochemistry for proliferative cell nuclear antigen (PCNA) can be performed. The sections can be deparaffinized with xylene and hydrated with serial concentrations of 100, 95, 80 and 60% ethanol. Endogenous peroxidase activity can be blocked with 0.6% hydrogen peroxide in methanol for 1 hour. Following three washes in tris-buffered saline (TBS)/0.1% BSA, nonspecific binding of antibody can be blocked with 1.5% normal horse serum (Vector Laboratories, Burlingame, Calif.) in TBS/0.5% BSA for 15 minutes at room temperature. The sections can then be incubated with mouse monoclonal antibodies against PCNA (Sigma) diluted at 1:1000 overnight at room temperature in a humidified chamber. After three washes in TBS/0.1% BSA, sections can be incubated with biotinylated horse anti-mouse IgG (Vector Laboratories) for 1 hour at room temperature. Following three washes in TBS/0.1% BSA, avidin-biotin complex (Vector Laboratories) can be applied for 30 minutes at room temperature. After extensive washes, the sections can be reacted with diaminobenzidine (DAB) (Sigma) for 2 minutes, then dehydrated and mounted. Mouse IgG (Sigma) can be used as a control primary antibody. The total number of cells and the number of positive stained cells can be counted with three random rectangles for each of the four selected injection sites per tibiae sample. The positive ratio for each site (e.g., proliferation index) can be calculated by dividing the number of positive stained cells by the total number of cells.

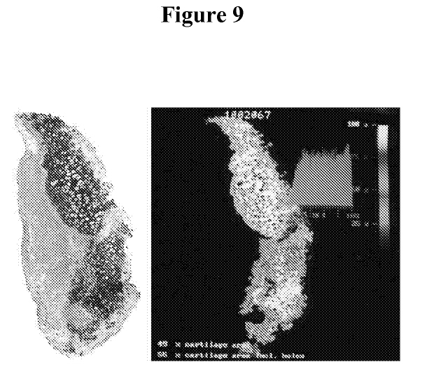

Histology and Histomorphometric Analysis: Specimens to be assessed histologically can be fixed for 2 days in 10% buffered formalin, 2 days in Bouin's solution, followed by decalcification in a 10% acetic acid, 0.85% NaCl, 10% formalin solution (AFS). This fixation/decalcification protocol can provide adequate decalcification, without loss of antigenic sites for immunohistochemistry. Then, the tissues can be embedded in paraffin, and sections 3 μm thick can be cut from the center of each block. Sections to be assessed for bone formation can be stained with Masson's trichrome. Sections to be assessed for cartilage formation can be stained with safranin O/fast green. One section from each of the periosteal injection sites and cultured periosteal explants can be analyzed by a computerized image analysis system (VIDAS 2.1 by Kontron Electronik, customized by Carl Zeiss Canada, Don Mills, Ontario) to determine the percent cross-sectional area that stained red with safranin O (FIG. 9). This measurement can represent the distribution of cartilage proteoglycan in the section and can be referred to as % cartilage. A section from each defect/repair site can be assessed according to a scoring system. Using a scoring system (Table 5), a representative section from each defect/repair site can be examined to determine the nature of the predominant tissue, affinity of its matrix for the safranin O stain, surface regularity, structural integrity, bonding to the adjacent articular cartilage, and the surface level of the newly formed tissue compared with that of the adjacent cartilage.

TABLE 5
A representative section from each defect/repair site can be examined
and scored according to the nature of the predominant tissue, affinity
of its matrix for the safranin O stain, surface regularity, structural
integrity, bonding to the adjacent articular cartilage, and the surface
level of the newly formed tissue compared with that of the
adjacent cartilage.
HISTOLOGICAL AND HISTOCHEMICAL GRADING SCALE

| | Score |
|---|---|
| Nature of the predominant tissue | |
| Cellular morphology | |
| Hyaline articular cartilage | 4 |
| Incompletely differentiated mesenchyme | 2 |
| Fibrous tissue or bone | 0 |
| Safranin-O staining of the matrix | |
| Normal or nearly normal | 3 |
| Moderate | 2 |
| Slight | 1 |
| None | 0 |
| Structural characteristics | |
| Surface regularity | |
| Smooth and intact | 3 |
| Superficial horizontal lamination | 2 |
| Fissures - 25 to 100 percent of the thickness | 1 |
| Severe disruption, including fibrillation | 0 |
| Structural integrity | |
| Normal | 2 |
| Slight disruption, including cysts | 1 |
| Severe disintegration | 0 |
| Thickness | |
| 100 percent of normal adjacent cartilage | 2 |
| 50-100 percent of normal cartilage | 1 |
| 0-50 percent of normal cartilage | 0 |
| Bonding to the adjacent cartilage | |
| Bonded at both ends of graft | 2 |
| Bonded at one end, or partially at both ends | 1 |
| Not bonded | 0 |
| Freedom from cellular changes of degeneration | |
| Hypocellularity | |
| Normal cellularity | 3 |
| Slight hypocellularity | 2 |
| Moderate hypocellularity | 1 |
| Severe hypocellularity | 0 |
| Chondrocyte clustering | |
| No clusters | 2 |
| <25 percent of the cells | 1 |
| 25-100 percent of the cells | 0 |
| Freedom from degenerative changes in adjacent cartilage | |
| Normal cellularity, no clusters, normal staining | 3 |
| Normal cellularity, mild clusters, moderate staining | 2 |
| Mild or moderate hypocellularity, slight staining | 1 |
| Severe hypocellularity, poor or no staining | 0 |

Periosteal Explant Culture: Periosteal explants, 3.5×3.5 mm can to be taken from the medial side of the proximal tibia of 6 or 12 month old New Zealand white rabbits using sharp subperiosteal dissection in the region of the injections sites (FIG. 11). Periosteal explants can be obtained within 15 minutes of death to control for post-mortem effects on chondrogenic potential. Immediately after surgical harvesting, the periosteal explants can be placed in Dulbecco's Modified Eagle Media (DMEM), 10% FBS with penicillin/streptomycin and 1 mM proline at 4° C. for no more than 1.5 hours prior to placement into culture wells. Twenty-four-well flat bottom culture plates can be prepared using previously published techniques. The medium above the gel layer can be replaced every second day. 50 μg/mL Vitamin C can be added every other day. 10 ng/mL TGF-β1 can be added for the first two days of culture. Cultures can be maintained for 6 weeks at 37° C. and 5% CO₂ and 95% air. In some case, no additional growth factor supplements can be added.

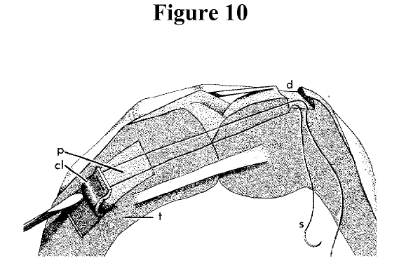

Autologous Periosteal Transplantation: Rabbits can be anesthetized, and one knee joint in each can be prepared for surgery. At the time of arthrotomy of one knee joint (randomly selected) of each rabbit, the patella can be dislocated laterally and a full-thickness transverse defect, measuring 5 mm from proximal to distal and traversing the entire width of the patellar groove, can be created using a coping saw and a rongeur (FIG. 10). In contrast to the concave transverse contour of the patellar groove, the base of the defect can be flat and can be below the level of the plate of cortical subchondral bone. The defect can extend to 2 mm below the surface of the adjacent cartilage in the middle of the patellar groove, whereas at the medial and lateral edges the depth can average 4-5 mm. A rectangular graft of periosteum, measuring 7×15 mm, can be obtained from the medial side of the proximal end of the tibia, using sharp subperiosteal dissection in order to include its cambium layer. The free periosteal graft can then be inverted and placed in the defect so that its cambium layer can be facing up into the joint. After the medial and lateral ends of the graft have been sutured to the periosteum on each side of the femur, the patella can be relocated, the arthrotomy wound can be closed with continuous locking 4-0 Vicryl suture, and the skin incision can be sutured with a continuous subcuticular 5-0 Dexon-S suture. Post-op, the rabbits can be allowed to move freely within their cages for 6 weeks, 6 months, or 1 year.

Mechanical Testing: Biopsies of repair cartilage can be analyzed by indentation testing. This can be performed on 3.5×5 mm diameter cylinders of repair cartilage harvested from the central portion of the repair tissue using a 3.5 mm dermal punch. The mechanical properties of the repair tissue can be compared to normal osteochondral plugs obtained from the contralateral knee. The specimen can be placed, unconstrained, on a flat dish with the cartilage surface perpendicular to the 1.6 mm cylindrical indenter. Loading can be applied at a rate of 5N/minute to a maximum force of 5N (maximum applied stress=2.5 MPa). A Dynamic Mechanical Analyzer (TA Instruments, New Castle, Del.) can be used to apply the loading. The 5N load limit can compress the cartilage beyond the toe region of the stress-strain curve. During testing the specimens can be immersed in saline. Applied stress can be calculated by dividing the applied force by the area of the indenter. Strain can be defined as the indentation displacement divided by the original cartilage thickness. Elastic modulus can calculated as the slope of the linear region of the stress-strain curve between 0 and 0.5 MPa applied stress and less than 20% strain.

Collagen Typing: Quantitative collagen typing can be performed in an automated fashion using the PhastSystem gel electrophoresis system (Pharmacia-LKB Biotechnology Group, Baie d'Urfé, Québec, Canada) and microgram sized samples. A 1 μL volume of sample, 8 μg/μL in sample buffer, can be applied to and separated on 20% homogeneous SDS-PAGE Phast-Gels. The gels, can be scanned using an LKB laser densitometer (Pharmacia-LKB Biotechnology Group), and the absorbance curves can be integrated with a computer software package (GelScan, Pharmacia-LKB Biotechnology Group, Canada). Percent type II collagen can be determined.

The invention will be further described in the following examples, which do not limit the scope of the invention described in the claims.

### EXAMPLES

### Example 1

### Developing Periosteum for Articular Cartilage Tissue Engineering

TGF-β1 and IGF-1, both separately and in combination, dramatically stimulate cartilage production in cultured periosteal explants. Studies described elsewhere demonstrated that TGF-β regulates IGF-1 autocrine/paracrine axis genes in chondrocytes and osteoblasts (Ortiz et al., J. Bone Miner. Res., 18:1066-72 (2003); Tsukazaki et al., Exp. Cell Res., 215:9-16 (1994)). The bioavailability of IGF-1 is regulated by IGF-1 binding proteins (IGFBPs) such as IGFBP-4. Pregnancy associated plasma protein-A (PAPP-A) is an IGF-independent-IGFBP-4 protease that cleaves IGFBP-4 thereby enhancing IGF-1 bioavailability. The effects of TGF-β1 on IGF-1, IGF-1 receptor, and IGF binding protein (IGFBPs) gene expression were investigated in cultured rabbit periosteal explants. It was also determined whether TGF-β1 can regulate IGFBP-4 protease activity and PAPP-A specifically in periosteal explants.

As determined by real-time PCR techniques, TGF-β1 treatment resulted in decreased IGF-1, IGF-1 receptor, and IGFBP-4 mRNA levels throughout periosteal chondrogenesis. Western ligand blots confirmed the regulation of IGFBP-4 protein and suggest that IGFBP-2, 3 and 5 levels are down regulated by TGF-β1 treatment. Consistent with these results, immunohistochemical staining for IGF-1 was also decreased by TGF-β1 treatment, which may reflect both decreased IGF-1 production and decreased IGF-1 binding.

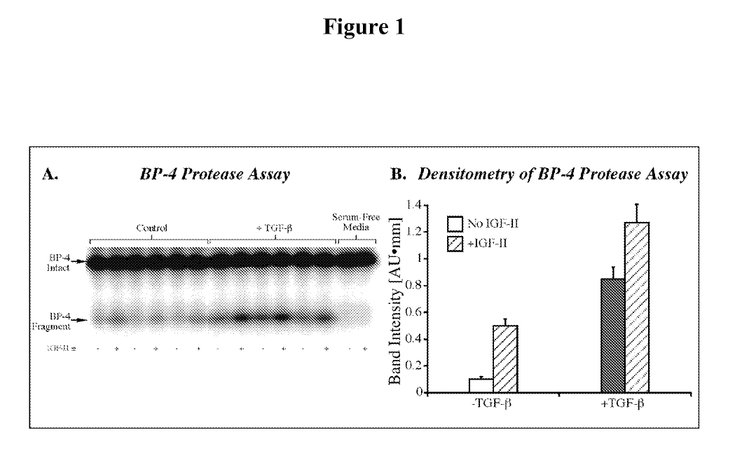

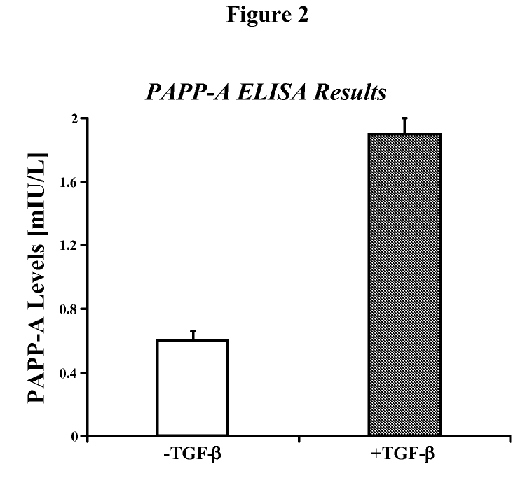

TGF-β1 treatment significantly increased IGFBP-4 protease activity present in conditioned medium from cultured periosteal explants (FIG. 1). Interestingly, TGF-β1 treatment significantly increased PAPP-A mRNA levels in periosteal explants cultured for 7 and 10 days. ELISA results confirmed that periosteal conditioned medium contained significantly lower levels of PAPP-A protein (FIG. 2). In addition, Western ligand blots demonstrated that the conditioned medium from TGF-β1 treated explants contained decreased amounts intact IGFBP-4 protein. Taken together, these results suggest that TGF-β1 can regulate the bioavailability of IGFBP-4 in periosteum by increasing its proteolysis by PAPP-A, thereby increasing IGF-1 bioavailability. This increased bioavailability might help to explain the observed additive effects of TGF-β1 and IGF-1 combined on periosteal cell proliferation and chondrogenesis.

These results demonstrate that TGF-β1 regulates the expression of multiple genes involved in the IGF-1 autocrine/paracrine axis during periosteal chondrogenesis. Continued examination of this signaling pathway during periosteal chondrogenesis can increase the understanding of fracture healing and augment the use of periosteal tissue in articular cartilage repair.

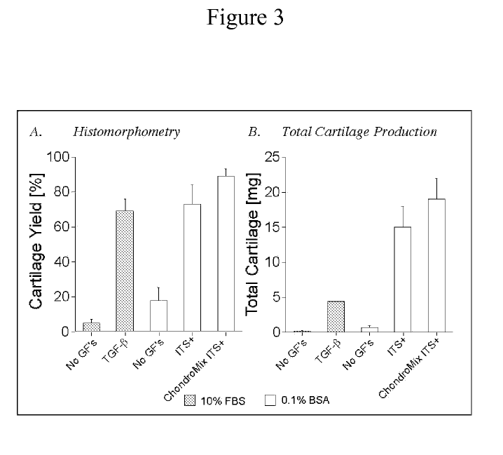

After testing a number of growth factor combinations in pilot studies, the combination of 10 ng/mL TGF-β1, 50 ng/mL bFGF, 5 μg/mL GH with ITS+(2.08 μg/mL insulin, transferrin selenious acid, 1.78 μg/mL linoleic acid, and 0.42 mg/mL bovine serum albumin) referred to as “ChondroMix” (Fitzsimmons J S, Sanyal A, Gonzalez C, Fukumoto T, Clemens V R, O'Driscoll S W, and Reinholz G G, Serum-Free Media For Periosteal Chondrogenesis In Vitro, J. Orthop. Res.), was further explored. The most significant effect of ChondroMix/ITS+ was observed on the overall growth of tissue, which impacted the total mass of cartilage formed (as opposed to the percentage of the tissue area that was cartilage; FIG. 3). The explants treated with ChondroMix (TGF-β1, bFGF, GH) and ITS+ with 0.1% BSA yielded an average of 19±3 mg of cartilage. This was significantly greater than the positive control explants (cultured in TGF-β1 in 10% FBS) which yielded 4±1 mg of cartilage (p<0.05). The explants treated with 0.3% ITS+ only (i.e., no growth factors and no serum) produced 15±3 mg of cartilage, which was significantly higher than the positive controls (TGF-β1 in 10% FBS; p<0.05), but not significantly different from the ChondroMix/ITS+ group (p>0.05). The negative control groups treated only with FBS or BSA without growth factors produced significantly less total cartilage mass (0.1±0.1 mg and 0.6±0.3 mg of cartilage, respectively) than the experimental groups or the positive control group (p<0.05).

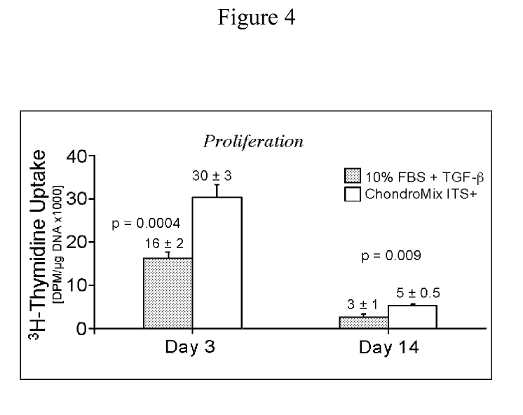

Measurements of tritiated thymidine uptake during the peak period of cell proliferation on day three (O'Driscoll and Fitzsimmons, Clin. Orthop., S190-207 (2001)) confirmed the effect of ChondroMix/ITS+ on tissue growth. ³H-thymidine uptake in the explants treated with ChondroMix/ITS+ (30,000±2,700 dpm/μg DNA) was significantly higher than that in our standard model cultured in 10% FBS with TGF-β1 added for the first 2 days (16,000±1,500 dpm/μg DNA; p<0.002). Tritiated thymidine uptake on day 14 was also significantly higher on day 14 with ChondroMix/ITS+ (5,300±500 dpm/μg DNA) higher than that in our standard model cultured in 10% FBS with TGF-β1 added for the first 2 days (2,700±700 dpm/μg DNA; p<0.02; FIG. 4). These data suggest that the stimulatory effect of ChondroMix/ITS+ on total cartilage growth may be due to sustained stimulation of cell proliferation.

A biosynthetic composite was developed for tissue engineering using periosteum and tantalum. A porous scaffold made of tantalum has been developed for potential application in reconstructive orthopedics and other surgical disciplines (Bobyn et al., J. Bone Joint Surg. Br., 81:907-14 (1999); Bobyn et al., J. Arthroplasty, 14:347-54 (1999)). The material has a high and interconnected porosity with a regular pore shape and size. The material can be made into complex shapes and used either as a bulk implant or as a surface coating.

Experiments were conducted to determine whether porous tantalum can be used as a primary support and shape specific interface between a periosteal explant and subchondral bone allowing biological resurfacing mediated by trabecular metal. First, it was determined whether periosteal tissue is compatible with the porous tantalum. Specifically, it was determined whether the porous tantalum would interfere with or support the normal growth and the chondrogenic differentiation of periosteal tissue. The feasibility of developing a biologic/prosthetic composite implant using a porous tantalum scaffold was evaluated, and the chondrogenic potential of periosteum was determined using an in vitro culture model.

Periosteum/tantalum composites were formed by attaching periosteal explants, harvested from the medial proximal tibiae of 2 two-month-old New Zealand white rabbits, onto porous tantalum cylinders (3 mm diameter×5 mm long). In initial experiments, the periosteal explants were carefully sutured to the tantalum. Subsequently, it was discovered that use of a suture was not necessary for the in vitro experiments because the explants remained attached to the tantalum in a manner similar to Velcro after simply placing the explants onto the tantalum. Control explants and composites were cultured in vitro with DMEM, 1% agarose, 10% fetal bovine serum with or without additional supplements.

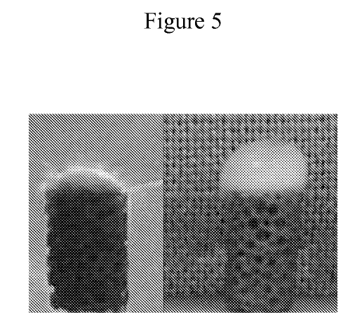

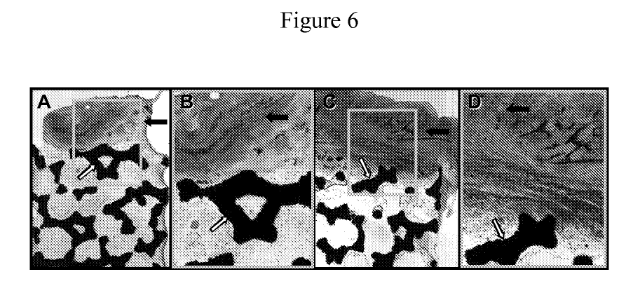

After six weeks of culture, the periosteal explants were well attached to the porous tantalum. Outgrowth of neocartilage was observed in the composites with the cambium layer of the periosteum facing away from the tantalum, whereas no neocartilage outgrowth was observed in the composites with the cambium layer facing the tantalum (FIG. 5). The cartilage yield obtained from the composites was comparable to the cartilage yield from control periosteal explants (explants cultured without tantalum). Ingrowth of tissue was observed in all of the composites regardless of the orientation of the periosteum (FIG. 6). The majority of the tissue ingrowth was fibrous in nature; however, some positive staining for cartilage was also observed within the tantalum. The nature of the tissue outgrowth was dependent on the orientation of the periosteum. Specifically, neocartilage outgrowth was only observed in composites containing periosteum with the cambium layer facing up away from the tantalum. These results support findings demonstrating that the cambium layer of the periosteum is the location of chondrocyte precursor cells (Ito et al., Osteoarthritis Cartilage, 9:215-223 (2001)).

In order to further evaluate the matrix production in the periosteum/tantalum composites, collagen typing analysis was performed. No statistically significant difference in the relative amount of type II collagen with respect to type I was observed in explants treated with TGF-β1 either in presence or absence of tantalum (p=0.9). The relative amount of collagen type II produced in the TGF-β1 treated composites and positive control explants was significantly greater than in the negative control explants (no growth factor treatment).

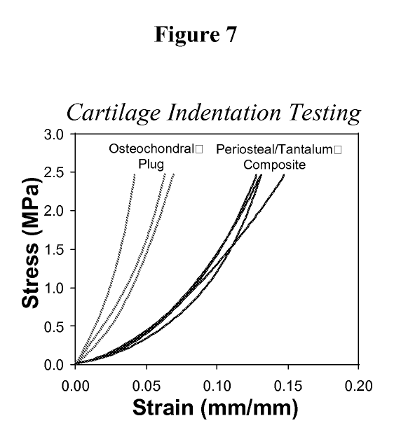

The mechanical properties of the periosteum/tantalum composites were examined using indentation analysis and compared to the properties of normal osteochondral plugs taken from the femoral condyles of rabbits. The shapes of the strain/stress curves produced from the tantalum/periosteal composites resemble the curve shapes obtained with the normal osteochondral plugs (FIG. 7). The tantalum/periosteal composite curve is displaced to the right, however, reflecting less stiffness than the normal osteochondral plug. It is important to note that after 6 weeks of culture, the composites contained less mature cartilage (50% positive staining for cartilage) compared to mature cartilage in the normal osteochondral plug. In addition, the tantalum was only filled with fibrous tissue after culturing the composites for six weeks in vitro. Overall, the composites used in this experiment were not developed to the stage expected after full integration into an osteochondral defect. The mechanical properties of a fully developed periosteum/tantalum composite are, therefore, compared to a normal osteochondral plug.

The results of these experiments demonstrate that under in vitro chondrogenic conditions, periosteum is compatible with porous tantalum. Because periosteal growth and chondrogenesis are not hindered by the presence of the tantalum when the periosteum is stitched with the cambium layer facing away from the tantalum, biological resurfacing of large osteochondral defects may be feasible using a porous tantalum/autologous periosteal construct. In addition, the robust ingrowth of periosteal tissue may further enhance the healing of large osteochondral defects by enabling firm attachment and integration of the periosteal explant.

### Example 2

### Rejuvenation Of Periosteal Chondrogenesis Using Local Growth Factor Injection

### Methods and Materials

A total of 367 New Zealand white rabbits (6, 12, and 24+ month-old) received subperiosteal injections of growth factors or vehicle in the medial side of the proximal tibia. After 1, 3, 5, or 7 days, the rabbits were sacrificed, and periosteum containing the injection sites was harvested together with the underlying bone as an intact osteoperiosteal specimen for histology (to determine cambium cellularity and thickness), or periosteal explants were elevated from the bone at the injection sites and cultured for 6 weeks (to determine in vitro cartilage forming capacity). Due to limited availability of the older animals, only in vitro cartilage formation was analyzed in the 24+ month-old rabbits. The rabbit ages were selected based on previous studies demonstrating significantly decreased cambium cellularity and thickness, and in vitro cartilage forming capacity in periosteum from rabbits 6-24 months old compared to 2 month-old.

### Subperiosteal Injections

Under general anesthesia (IM Ketamine/Xylazine/Acepromazine at 50/5.0/0.75 mg/Kg), subperiosteal injections of growth factor or vehicle were performed percutaneously using a Hamilton syringe with a 30-gauge needle. As illustrated in FIG. 11, a single injection (10 μL) of the same vehicle or growth factor was made 5 mm proximal to the distal edge of the tibial tuberosity in each of four approximately 3.5×3.5 mm regions on each tibia (corresponding to the area that is normally used for periosteal harvesting in this model). One limb received the growth factor injection, and the contralateral limb received vehicle, alternating sides to eliminate potential bias. The 10 μL injections contained recombinant human TGF-β1 (20 or 200 ng) or vehicle (4 mM HCl, 1 mg/mL BSA), recombinant human IGF-1 (0.2 or 2.0 μg) or vehicle (10 mM acetic acid, 0.1% BSA), or a combination of 200 ng TGF-β1 and 2.0 μg IGF-1 or vehicle (5 mM acetic acid, 2 mM HCL, 0.1% BSA). The recombinant human TGF-β1 and recombinant human IGF-1 were purchased from R&D Systems, Inc. (Minneapolis, Minn.). Prior to initiation of the experiment, dye was injected in rabbit carcasses in order to establish the injection technique.

### Cambium Cellularity, Thickness, and Cell Density

Using a bone saw, periosteum containing the injection sites was harvested together with the underlying bone as an intact osteoperiosteal specimen from the medial proximal tibiae region. The samples were decalcified using EDTA. After decalcification, the individual injection sites were separated and individually paraffin embedded. Each sample was oriented “on edge” during the embedding process to yield cross-sections of the cambium layer and underlying bone. Samples were then sectioned at 5 μm thick and stained with haematoxylin and eosin (H & E). Cambium maximum thickness (μm), cellularity (cells/mm), and cell density (cells/cm²) were determined by a blinded technician as described elsewhere (Gallay et al. J. Orthop. Res., 12(4):515-25 (1994) and O'Driscoll et al., J. Orthop. Res., 19(1):95-103 (2001)) in a manner that was adapted for the KS300 computerized image analysis system (Carl Zeiss Vision GmbH, Hallbergmoos, Germany). Briefly, three sections were obtained from each tissue sample and scanned at the injection site using the Zeiss AuxioCam MRc at the same magnification with the periosteal layer line horizontal to the bottom edge of the scan's frame of reference. Each cambium layer was outlined by hand. Maximum thickness was determined by comparing thicknesses in the outlined area from top to bottom. For determining cellularity, a custom macro was used to detect, mark, and calculate the number of nuclei in the outlined areas based on color. The computer program determined the total area occupied by nuclei within the traced cambium layer and divided that by average nuclear size (determined separately). This value for the total number of cells within the cambium layer was then divided by the length of the cambium layer that was outlined. The result was the average number of cambium cells per mm. Cell density was determined by dividing the total number of cells within the cambium layer by the area of the outlined section (cells/cm²). Values obtain from sections made from the same tissue sample were then averaged to give overall mean values for cambium maximum thickness, cellularity, and cell density for each tissue sample.

### In Vitro Cartilage Formation

Periosteum was elevated as four 3.5×3.5 mm sections from the medial side of the proximal tibia of 6, 12, or 24+ month-old New Zealand white rabbits using sharp dissection in the regions of the injection sites (FIG. 11). All periosteal explants were obtained within 15 minutes of death to control for post-mortem effects on chondrogenic potential. The explants were then cultured as described elsewhere (O'Driscoll et al., J. Bone Joint Surg. Am., 76(7):1042-51 (1994)). Briefly, immediately after surgical harvesting, the periosteal explants were placed in Dulbecco's Modified Eagle Media (DMEM), 10% FBS with penicillin/streptomycin, and 1 mM proline at 4° C. for no more than 1.5 hours prior to transfer to 1% agarose culture. The medium was supplemented with 10 ng/mL TGF-β1 for the first two days of culture and 50 μg/mL Vitamin C through the duration of the culture period. The medium above the gel layer was replaced every second day. Cultures were maintained for 6 weeks at 37° C. and 5% CO₂ and 95% air. The explants were then weighed, embedded in paraffin, sectioned, and stained with Safranin O/fast green. Computerized histomorphometry was then used by a blinded technician to determine the cartilage yield (% area) and total cartilage index (% area×wet weight=mg total cartilage) in each specimen (O'Driscoll et al., Tissue Eng., 5:13-23 (1999) and Fitzsimmons et al., J. Orthop. Res., 22(4):716-25 (2004)).

### Statistical Analysis

Data were expressed as mean±S.D (tables) or S.E.M. (bar graphs). Statistical differences between each treatment group and corresponding vehicle control were evaluated using one-way Analysis of Variance (ANOVA). Statistical differences between treatment groups were evaluated using the Least Squares Means Differences Student's t-Test (p=0.05).

### Results

### Cambium Cellularity, Thickness and Cell Density

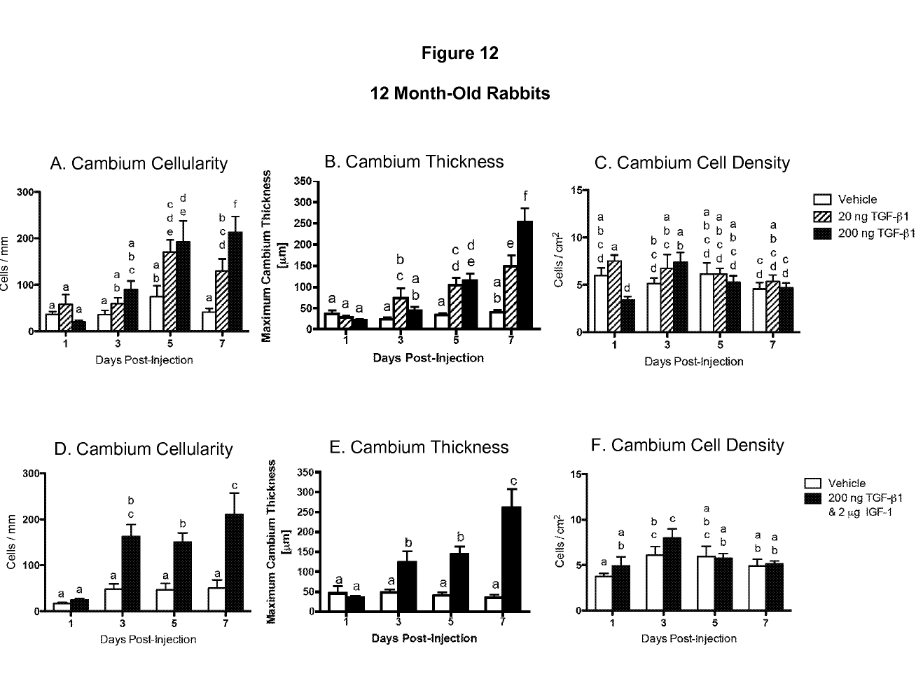

Injection of TGF-β1 with or without IGF-1 increased cambium cellularity and maximum cambium thickness in both the 6 and 12 month-old rabbits (FIG. 12 and Table 6). In the 12 month-old rabbits, cambium cellularity and maximum cambium thickness values were dependent on the duration of the treatment (p<0.0001) and the dose (p<0.0001). Peak cambium cellularity (213±118 cells/mm, n=12) and maximum cambium thickness (252±120 μm, n=13), were observed in the 200 ng TGF-β1 group 7 days post-injection (FIG. 12 and Table 6). In the 12 month-old 200 ng TGF-β1 plus 2.0 μg IGF-1-injected rabbits, the peak cambium cellularity (211±154 cells/mm, n=11) and maximum cambium thickness (260±177 μm, n=14), also occurred at 7 days post-injection (FIG. 12 and Table 6). Cambium cell density remained steady over time, and no significant difference was found in any of the 12 month-old growth factor injected rabbits compared to the vehicle controls (FIG. 12 and Table 6).

TABLE 6
Cambium Cellularity, Thickness and Cell Density

| Days Post-Injection | |
|---|---|
| Day 1 | Day 3 |

| | Age | | 20 ng | 200 ng | | 20 ng | 200 ng |
|---|---|---|---|---|---|---|---|
| | (Months) | Vehicle | TGF-β1 | TGF-β1 | Vehicle | TGF-β1 | TGF-β1 |
| Cellularity | 6 | 89 ± 47^{a,b} | 40 ± 40^{a} | 127 ± 56^{a-c} | 89 ± 48^{a,b} | 232 ± 79^{c-e} | 149 ± 79^{b,c} |
| (cells/mm) | 12 | 36 ± 32^{a} | 58 ± 75^{a} | 20 ± 9^{a} | 35 ± 43^{a} | 59 ± 36^{a,b} | 89 ± 61^{a-c} |
| Thickness | 6 | 88 ± 53^{a,b} | 60 ± 21^{a} | 157 ± 73^{b-e} | 98 ± 85^{a,b} | 132 ± 42^{a-d} | 162 ± 84^{c-e} |
| (μM) | 12 | 36 ± 43^{a} | 28 ± 16^{a} | 21 ± 12^{a} | 24 ± 21^{a} | 74 ± 75^{b,c} | 43 ± 36^{a,b} |
| Cell Density | 6 | 5 ± 2.2^{a-d} | 3 ± 1.7^{a-c} | 5 ± 2.2^{a-d} | 5 ± 2.3^{c-e} | 8 ± 0.7^{f} | 5 ± 0.5^{c-e} |
| (cell/cm²) | 12 | 6 ± 3.9^{a-d} | 8 ± 2.3^{a} | 3 ± 0.9^{d} | 5 ± 2.6^{b-d} | 7 ± 4.1^{a-c} | 7 ± 3.4^{a,b} |

| Days Post-Injection | |
|---|---|
| Day 5 | Day 7 |

| | Age | | 20 ng | 200 ng | | 20 ng | 200 ng |
|---|---|---|---|---|---|---|---|
| | (Months) | Vehicle | TGF-β1 | TGF-β1 | Vehicle | TGF-β1 | TGF-β1 |
| Cellularity | 6 | 52 ± 38^{a} | 188 ± 122^{c} | 130 ± 86^{a-c} | 93 ± 66^{a,b} | 165 ± 108^{b,c} | 153 ± 153^{b,c} |
| (cells/mm) | 12 | 74 ± 89^{a,b} | 170 ± 73^{c-e} | 192 ± 111^{d,e} | 41 ± 40^{a} | 129 ± 99^{b-d} | 213 ± 118^{f} |
| Thickness | 6 | 113 ± 85^{a-c} | 200 ± 77^{e} | 208 ± 88^{e} | 108 ± 77^{a,b} | 279 ± 100^{f} | 185 ± 134^{d,e} |
| (μM) | 12 | 33 ± 26^{a} | 104 ± 63^{c,d} | 115 ± 63^{d,e} | 40 ± 27^{a,b} | 149 ± 91^{e} | 252 ± 120^{f} |
| Cell Density | 6 | 3 ± 1.6^{a} | 5 ± 2.5^{c,d} | 4 ± 1.3^{a,b} | 5 ± 2.4^{b-d} | 5 ± 1.3^{a-d} | 4 ± 1.8^{a-c} |
| (cell/cm²) | 12 | 6 ± 4.3^{a-d} | 6 ± 1.7^{a-d} | 5 ± 1.7^{a-d} | 5 ± 3.4^{c,d} | 5 ± 2.6^{a-d} | 5 ± 1.7^{c-d} |

| | Age | | 200 ng TGF-β1 | | 200 ng TGF-β1 | | 200 ng TGF-β1 | | 200 ng TGF-β1 |
|---|---|---|---|---|---|---|---|---|---|
| | (Months) | Vehicle | & 2 μg IGF-1 | Vehicle | & 2 μg IGF-1 | Vehicle | & 2 μg IGF-1 | Vehicle | & 2 μg IGF-1 |
| Cellularity | 6 | 66 ± 31^{a,b} | 136 ± 113^{b,c} | 98 ± 52^{a-c} | 160 ± 75^{c,d} | 55 ± 17^{a} | 230 ± 111^{e} | 86 ± 47^{a,b} | 226 ± 126^{d,e} |
| (cells/mm) | 12 | 17 ± 5.9^{a} | 25 ± 11^{a} | 48 ± 37^{a} | 163 ± 90^{b,c} | 46 ± 47^{a} | 150 ± 74^{b} | 50 ± 57^{a} | 211 ± 154^{c} |
| Thickness | 6 | 116 ± 78^{a,b} | 147 ± 92^{b} | 126 ± 78^{b} | 125 ± 52^{a,b} | 77 ± 45^{a} | 198 ± 66^{c} | 103 ± 64^{a,b} | 206 ± 77^{c} |
| (μM) | 12 | 46 ± 73^{a} | 34 ± 20^{a} | 47 ± 33^{a} | 124 ± 106^{b} | 40 ± 30^{a} | 145 ± 71^{b} | 35 ± 27^{a} | 260 ± 177^{c} |
| Cell Density | 6 | 5 ± 1.8^{a} | 5 ± 2.1^{a} | 5 ± 2.6^{a} | 6 ± 2.8^{a} | 5 ± 2.4^{a} | 6 ± 1.6^{a} | 5 ± 1.9^{a} | 6 ± 2.4^{a} |
| (cell/cm²) | 12 | 4 ± 1.2^{a} | 5 ± 3.3^{a,b} | 6 ± 3.0^{b,c} | 8 ± 3.5^{c} | 6 ± 3.7^{a-c} | 6 ± 1.8^{a,b} | 5 ± 2.4^{a,b} | 5 ± 1.1^{a,b} |

The data are presented as means ± S.D. Lowercase letters indicate the results of post-hoc testing using Student's test (p < 0.05). Values in each row with letters in common are not statistically different from one another.

In the 6 month-old TGF-β1-injected rabbits, cambium cellularity and maximum cambium thickness values were dependent on the duration of the treatment (p<0.0001) and the dose (p<0.02). The peak cambium cellularity (232±79 cells/mm, n=3) was observed in the 20 ng TGF-β1 group 3 days post-injection while the maximum cambium thickness (279±100 μm, n=13), was found in the 20 ng TGF-β1 group 7 days post-injection (Table 6). In the 6 month-old 200 ng TGF-β1 plus 2.0 μg IGF-1-injected rabbits, cambium cellularity and maximum cambium thickness values were only dependent on treatment (p<0.0001) not duration. In the combined treatment, peak cambium cellularity (230±111 cells/mm, n=15) occurred at 5 days post-injection while peak maximum cambium thickness (206±77 μm, n=15) was observed at 7 days post-injection (Table 6). In the 6 month-old rabbits, a significant increase in cell density (p<0.05) compared to vehicle injection was only observed in the 20 ng TGF-β1-injected rabbits 1 day post-injection (Table 6).

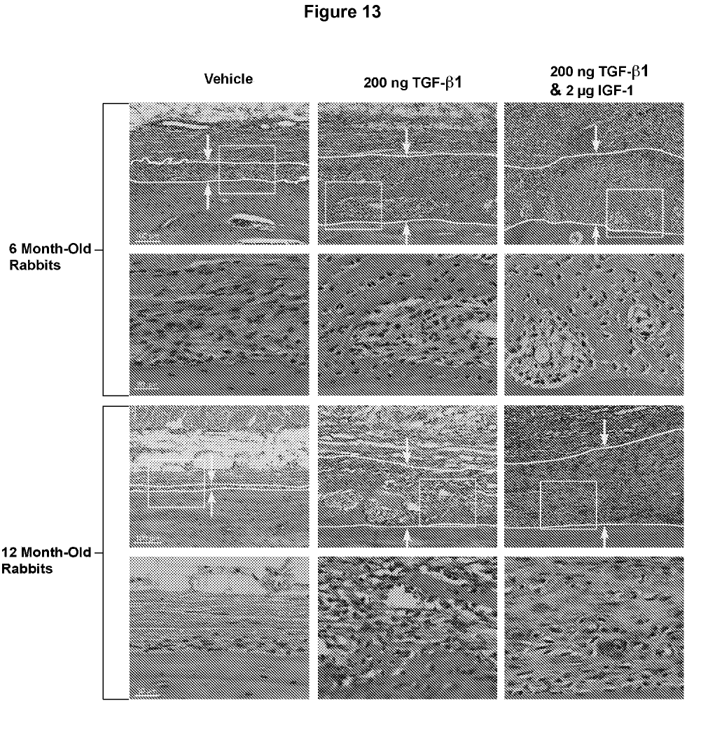

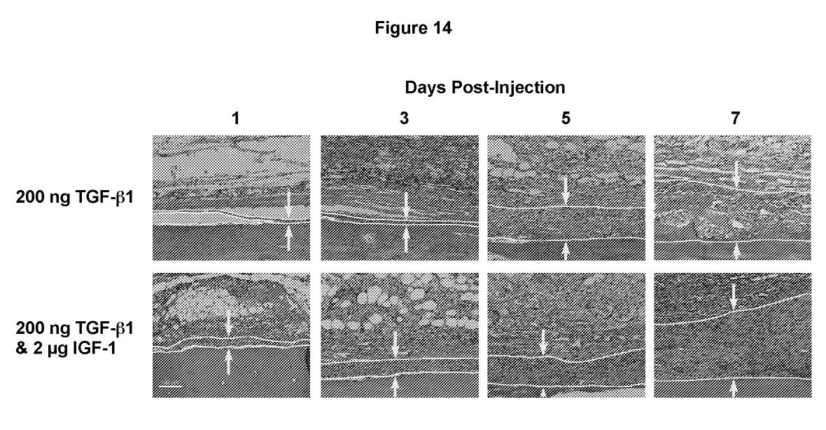

FIG. 13 shows examples of typical (representative of the means) H & E stained histological sections of periosteum on bone from the 200 ng TGF-β1 and the 200 ng TGF-β1 plus 2.0 μg IGF-1 combined treatment groups compared to vehicle controls at 7 days post-injection. In the vehicle control group, the periosteum in the 6 month-old rabbits was clearly thicker than in the 12 month-old rabbits. The mean maximum cambium thickness of the vehicle-injected limbs was significantly higher in the 6 month-old rabbits (117±84 μm, n=304) compared to the 12-month-old rabbits (33±30 μm, n=287), p<0.0001 (data pooled from all time points) demonstrating the age-related thinning of the periosteum in the experimental rabbits. Thickening of the periosteum was apparent in both the 200 ng TGF-β1, and the 2.0 μg IGF-1 plus 200 ng TGF-β1 groups compared to the vehicle controls. In addition, increased extracellular matrix was visible within the cambium of both growth factor treated groups. FIG. 14 illustrates the effect of 200 ng TGF-β1, and the 2.0 μg IGF-1 plus 200 ng TGF-β1 injection in the 12 month-old rabbits over time. In the combined treatment group, thickening of the cambium was observed by day 3 post-injection, was apparent in both treatment groups by day 5, and increased further by day 7 post-injection. Increased extracellular matrix in the cambium was noted in both treatment groups by day 5 and increased by day 7 post-injection. Subperiosteal injection of IGF-1 (0.2 or 2.0 μg) had no significant effect on cambium layer thickness compared to vehicle controls in the 6 month-old rabbits at any of the time points. In the 12 month-old rabbits, a small, but statistically significant increase in the maximum cambium thickness was observed 7 days post-injection in both the 0.2 μg (44±69 μm, n=16) and 2.0 μg (36±22 μm, n=16) IGF-1 doses compared to vehicle controls (30±14 μm, n=32), p<0.05.

### In Vitro Periosteal Cartilage Formation

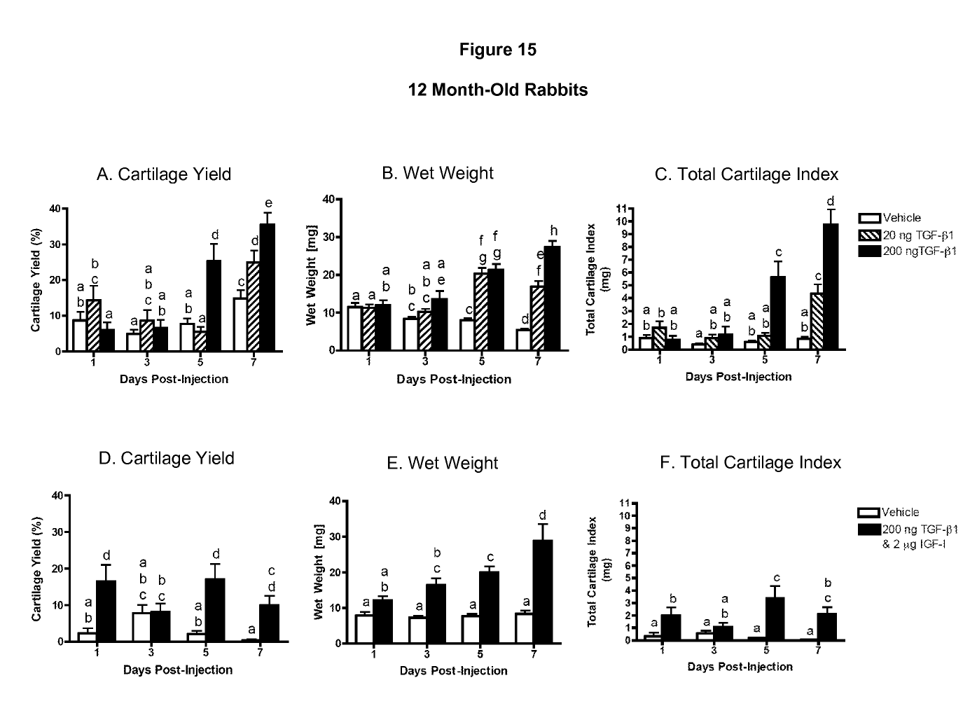

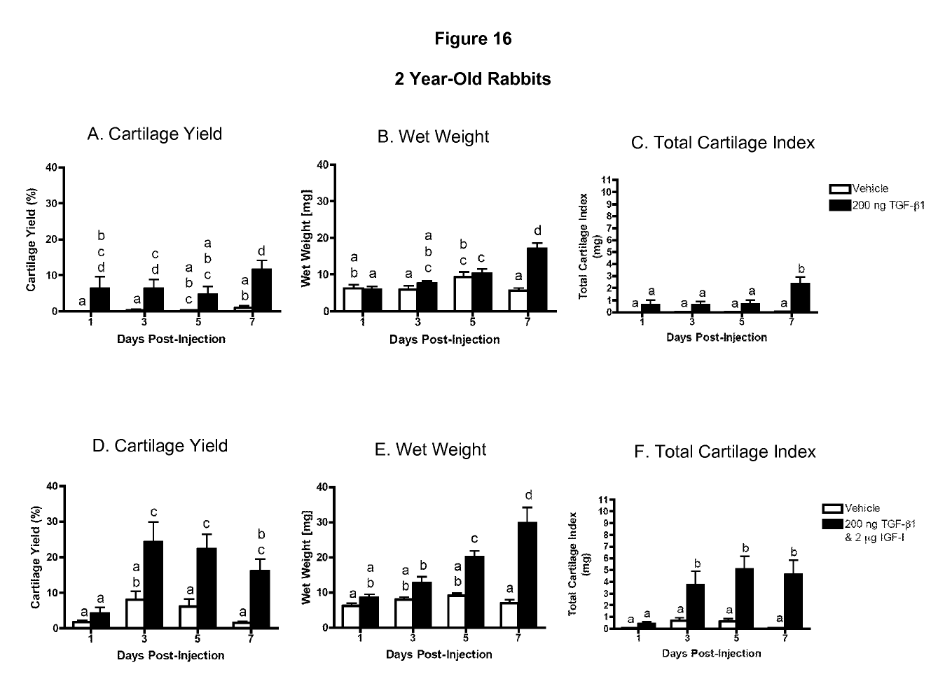

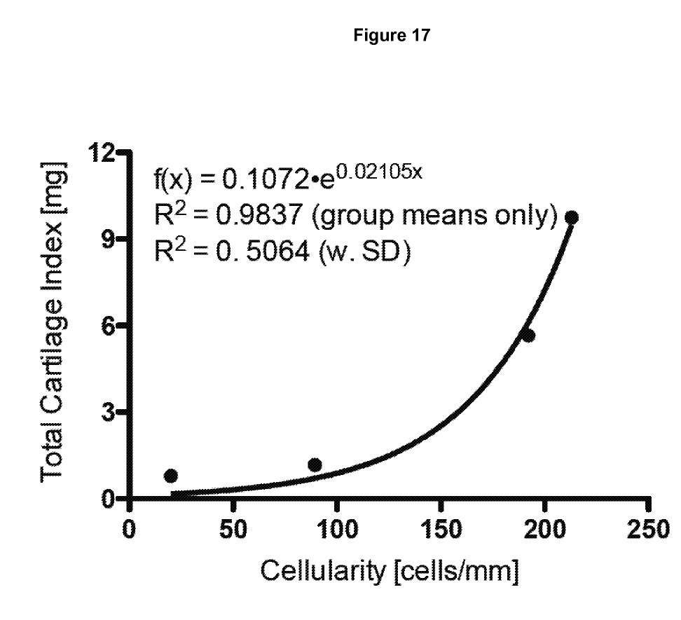

Injection of 200 ng TGF-β1 with or without 2.0 μg IGF-1 increased in vitro periosteal cartilage yield, explant wet weight, and total cartilage index in all age groups after six weeks of culture (FIG. 15-17 and Table 7). In the 12 month-old TGF-β1-treated rabbits, cartilage yield, explant wet weight, and total cartilage index values were dependent on dose and duration of the treatment (p<0.0001). Injection of 200 ng TGF-β1 significantly increased in vitro periosteal cartilage yield and total cartilage index when harvested 5 and 7 days post-injection (FIG. 15 and Table 7). Peak cartilage yield (36±14%, n=16) and explant wet weight (27±6.3 mg, n=16) in the 12 month-old rabbits were observed in the 200 ng TGF-β1 7-day post-injection group, resulting in a 12-fold increase in total cartilage index in the 200 ng TGF-β1 (9.7±4.7 mg, n=16) injected rabbits (p<0.0001) compared to vehicle controls (0.8±1.0 mg, n=32). As shown in FIG. 17, a strong correlation between the mean total cartilage index and cambium cellularity was observed in the 200 ng TGF-β1 injected rabbits. In the 12 month-old rabbits, wet weight values were dependent on both duration and treatment, whereas cartilage yield and total cartilage index were dependent on treatment only (p<0.0001). Injection of 200 ng TGF-β1 plus 2.0 μg IGF-1 significantly increased cartilage yield when the periosteum was explanted 1, 5, or 7 days post-injection (FIG. 15 and Table 7). The peak cartilage yield (17±17%, n=16) and peak total cartilage index (3.4±3.9 mg, n=16) values in the combined treatment groups were lower than the 200 ng TGF-β1 in the 12 month-old rabbits. However, when compared to vehicle-injected controls (0.16±0.32 mg, n=16), a comparable 20-fold increase in total cartilage index was observed. In the 2 year-old rabbits, 200 ng TGF-β1 injections significantly increased in vitro periosteal cartilage yield at all time points, and 200 ng TGF-β1 plus 2.0 μg IGF-1 injections increased cartilage yield from periosteum explanted 3, 5, and 7 days post-injection (FIG. 16 and Table 7). In the 2 year-old 200 ng TGF-β1-injected rabbits, explant wet weights (p<0.0001) and total cartilage index (p<0.004) were treatment and duration-dependent, whereas cartilage yield was only treatment dependent (p<0.0001). In the 2 year-old 200 ng TGF-β1 plus 2.0 μg IGF-1 combined treatment group, cartilage yield (p<0.0001), explant wet weight (p<0.0001), and total cartilage index (p<0.003) were duration and treatment dependent. In contrast to the 12 month-old rabbits, peak cartilage production was observed in the 200 ng TGF-β1 plus 2.0 μg IGF-1 combined treatment group. Peak cartilage yield (24±22%, n=16) in the 2 year-old rabbits was observed in periosteum explanted 3-days post-injection, and peak total cartilage index (5.1±4.4 mg, n=16) was found in periosteum explanted 5-day post-injection. In the 6 month-old TGF-β1-treated rabbits, cartilage yield (p<0.0001), and total cartilage index (p<0.04) values were dose and duration-dependent and explant wet weights (p<0.0001) were only dose dependent. Injection of 200 ng TGF-β1 significantly increased cartilage yield from periosteum explanted 1, 5 and 7 days post-injection in 6 month-old rabbits. Peak cartilage yield (33±19%, n=16) and total cartilage index (6.8±1.2, n=16) in the 6 month-old rabbits were observed in periosteum explanted 1-day after injection of 200 ng TGF-β1. In the combined 200 ng TGF-β1 plus 2.0 μg IGF-1 treated rabbits, total cartilage index (p<0.05), and explant wet weights (<0.02) were duration and treatment dependent. The combined treatment significantly increased cartilage yield from periosteum explanted 5 days post-injection (Table 7).

TABLE 7
Cartilage Yield, Wet Weight, and Total Cartilage Index

| Days Post-Injection | |
|---|---|
| Day 1 | Day 3 |

| | Age | | 20 ng | 200 ng | | 20 ng | 200 ng |
|---|---|---|---|---|---|---|---|
| | (Months) | Vehicle | TGF-β1 | TGF-β1 | Vehicle | TGF-β1 | TGF-β1 |
| Cartilage | 6 | 20 ± 17^{b-d} | 13 ± 14^{d-f} | 33 ± 19^{a} | 26 ± 20^{a-c} | 17 ± 12^{c-e} | 33 ± 19^{a-c} |
| Yield | 12 | 9 ± 13^{a,b} | 14 ± 16^{b,c} | 6 ± 9^{a} | 5 ± 7^{a} | 9 ± 12^{a-c} | 7 ± 9^{a,b} |
| (%) | 24 | 0.06 ± 0.26^{a} | NA | 6 ± 13^{b-d} | 0.4 ± 1.2^{a} | NA | 6 ± 10^{c,d} |
| Wet Weight | 6 | 11 ± 4.4^{a} | 12 ± 4.2^{a-c} | 19 ± 6.4^{d,e} | 14 ± 4.7^{b,c} | 18 ± 6.9^{d,e} | 15 ± 8.5^{c,d} |
| (mg) | 12 | 12 ± 6.0^{a} | 11 ± 3.7^{a} | 12 ± 5.1^{a,b} | 8 ± 3.2^{b,c} | 10 ± 2.9^{a-c} | 14 ± 8.8^{a,e} |
| | 24 | 6 ± 3.8^{a,b} | NA | 6 ± 3.8^{a} | 6 ± 4.3^{a} | NA | 8 ± 2.5^{a-c} |
| Total | 6 | 2.3 ± 0.40^{a,b} | 1.5 ± 0.44^{a,b} | 6.8 ± 1.2^{f} | 4.0 ± 0.60^{c-e} | 3.1 ± 0.59^{b-d} | 4.6 ± 0.94^{d,e} |
| Cartilage | 12 | 0.9 ± 1.4^{a,b} | 1.7 ± 2.0^{b} | 0.78 ± 1.1^{a,b} | 0.4 ± 0.57^{a} | 0.9 ± 1.1^{a,b} | 1.1 ± 2.5^{a,b} |
| Index (mg) | 24 | .006 ± 0.02^{a} | NA | 0.6 ± 1.6^{a} | 0.02 ± 0.06^{a} | NA | 0.6 ± 1.1^{a} |

| Days Post-Injection | |
|---|---|
| Day 5 | Day 7 |

| | Age | | 20 ng | 200 ng | | 20 ng | 200 ng |
|---|---|---|---|---|---|---|---|
| | (Months) | Vehicle | TGF-β1 | TGF-β1 | Vehicle | TGF-β1 | TGF-β1 |
| Cartilage | 6 | 10 ± 12^{e,f} | 13 ± 14^{d-f} | 29 ± 21^{a,b} | 6 ± 8^{f} | 9 ± 11^{e,f} | 18 ± 13^{c-e} |
| Yield | 12 | 8 ± 9^{a,b} | 6 ± 5^{a} | 25 ± 19^{d} | 15 ± 14^{c} | 25 ± 13^{d} | 36 ± 14^{e} |
| (%) | 24 | 0.3 ± 0.69^{a-c} | NA | 5 ± 9.0^{a-c} | 0.9 ± 2.5^{a,b} | NA | 12 ± 10^{d} |
| Wet Weight | 6 | 10 ± 4.6^{a} | 15 ± 8.2^{c,d} | 19 ± 6.9^{e} | 11 ± 6.4^{a,b} | 15 ± 5.0^{c,d} | 22 ± 7.7^{e} |
| (mg) | 12 | 8 ± 3.4^{c} | 20 ± 6.6^{f,g} | 21 ± 5.8^{f,g} | 5 ± 2.2^{d} | 17 ± 6.2^{e,f} | 27 ± 6.3^{h} |
| | 24 | 9 ± 5.7^{b,c} | NA | 10 ± 4.7^{c} | 6 ± 2.8^{a} | NA | 17 ± 6.4^{d} |
| Total | 6 | 1.1 ± 0.28^{a} | 2.2 ± 0.73^{a-c} | 5.5 ± 1.3^{e,f} | 0.9 ± 0.25^{a} | 1.2 ± 0.33^{a,b} | 4.3 ± 1.1^{d,e} |
| Cartilage | 12 | 0.6 ± 0.70^{a,b} | 1.1 ± 0.95^{a,b} | 5.7 ± 4.9^{c} | 0.8 ± 0.97^{a,b} | 4.4 ± 2.8^{c} | 9.7 ± 4.7^{d} |
| Index (mg) | 24 | 0.02 ± 0.05^{a} | NA | 0.7 ± 1.4^{a} | 0.06 ± 0.16^{a} | NA | 2.4 ± 2.2^{b} |

| | Age | | 200 ng TGF-β1 | | 200 ng TGF-β1 | | 200 ng TGF-β1 | | 200 ng TGF-β1 |
|---|---|---|---|---|---|---|---|---|---|
| | (Months) | Vehicle | & 2 μg IGF-1 | Vehicle | & 2 μg IGF-1 | Vehicle | & 2 μg IGF-1 | Vehicle | & 2 μg IGF-1 |
| Cartilage | 6 | 14 ± 15^{a} | 20 ± 20^{a,b} | 24 ± 23^{b} | 29 ± 23^{b} | 12 ± 12^{a} | 30 ± 22^{b} | 20 ± 16^{a,b} | 12 ± 12^{a} |
| Yield | 12 | 2 ± 5.8^{a,b} | 16 ± 18^{d} | 8 ± 9.2^{a-c} | 8 ± 9.0^{b,c} | 2 ± 3.5^{a,b} | 17 ± 17^{d} | 0.4 ± 1.2^{a} | 10 ± 10^{c,d} |
| (%) | 24 | 2 ± 1.7^{a} | 4 ± 6.9^{a} | 8 ± 9.1^{a,b} | 24 ± 22^{c} | 6 ± 8.6^{a} | 22 ± 17^{c} | 2 ± 1.7^{a} | 16 ± 14^{b,c} |
| Wet Weight | 6 | 15 ± 4.5^{a} | 14 ± 4.4^{a} | 17 ± 5.9^{a,b} | 22 ± 6.3^{b} | 12 ± 7.5^{a} | 30 ± 21^{c} | 14 ± 5.7^{a} | 30 ± 6.8^{c} |
| (mg) | 12 | 8 ± 3.9^{a} | 12 ± 4.3^{a,b} | 7 ± 2.3^{a} | 16 ± 8.0^{b,c} | 8 ± 2.8^{a} | 20 ± 6.8^{c} | 8 ± 4.2^{a} | 29 ± 19^{d} |
| | 24 | 7 ± 3.8^{a} | 9 ± 3.5^{a,b} | 8 ± 3.2^{a,b} | 13 ± 7.1^{b} | 9 ± 3.1^{a,b} | 20 ± 6.8^{c} | 7 ± 3.8^{a} | 30 ± 18^{d} |
| Total | 6 | 1.7 ± 1.7^{a} | 3.2 ± 3.0^{a,b} | 5.2 ± 5.2^{b,c} | 5.6 ± 4.8^{b,c} | 2.0 ± 3.1^{a} | 7.1 ± 7.2^{c} | 3.5 ± 3.4^{a,b} | 3.7 ± 3.7^{a,b} |
| Cartilage | 12 | 0.3 ± 1.1^{a} | 2.0 ± 2.6^{b} | 0.6 ± 0.8^{a} | 1.1 ± 1.3^{a,b} | 0.16 ± 0.32^{a} | 3.4 ± 3.9^{c} | 0.04 ± 0.18^{a} | 2.1 ± 2.3^{b,c} |
| Index (mg) | 24 | 0.08 ± 0.09^{a} | 0.4 ± 0.7^{a} | 0.7 ± 1.0^{a} | 3.7 ± 4.6^{b} | 0.6 ± 0.08^{a} | 5.1 ± 4.4^{b} | 0.09 ± 0.08^{a} | 4.6 ± 4.8^{b} |

The data are presented as means ± S.D. Lowercase letters indicate the results of post-hoc testing using Student's test (p < 0.05). Values in each row with letters in common are not statistically different from one another

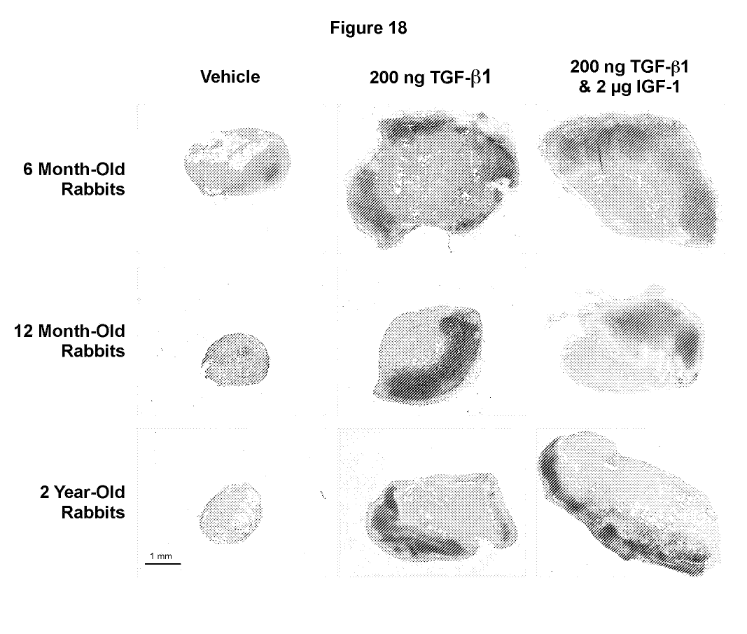

FIG. 18 contains typical (representative of the means) examples of safranin O/fast green stained sections of periosteal explants after in situ vehicle or growth factor injection followed by six weeks in culture. The mean cartilage yield produced by periosteal explants after six weeks of culture in the vehicle-injected periosteum was significantly higher in the 6 month-old rabbits (17.6±17.2%, n=319) compared to the 12-month-old rabbits (7.4±11.1%, n=318) and the 2 year-old rabbits (1.9±4.6%, n=188), p<0.0001 (data pooled from all time points) demonstrating an age-related decline in periosteal chondrogenesis in the experimental animals. FIG. 18 clearly illustrates the increase in cartilage formation, as detected by safranin O/fast green staining, observed after injection of 200 ng TGF-β1 alone or in combination with 2.0 μg IGF-1 followed by six weeks of culture. It is obvious from these examples that periosteal explant size was also increased. This observation is further illustrated by comparing the wet weights of the periosteal explants after the culture period (FIGS. 15 and 17 and Table 7). Subperiosteal injection of IGF-1 (0.2 or 2.0 μg) did not significantly increase cartilage yield, explant wet weights or total cartilage index compared to vehicle controls at any of the post-injection explant times in any age group. However, injection 200 ng TGF-β1 alone or in combination with 2 μg IGF-1 significantly increased wet weight in all age groups.

### Example 3

### Obtaining Periosteal Tissue Grafts In Vivo

The following was performed to determine the efficacy of in vivo rejuvenated periosteum for the regeneration of osteochondral tissue in vivo.

All TGF-β1 injections and operative procedures were performed under general anesthesia (50 mg/kg ketamine; 5 mg/kg xylazine; 0.75 mg/kg acepromazine). 12 month-old rabbits were used. In the rejuvenation group, rabbits received subperiosteal injection of 200 ng of TGF-β1 percutaneously (7 days prior to surgery), in four regions of the medial side of the proximal tibia spread evenly along the projected graft harvest site. The control rabbits received no injection prior to surgery.

The knee joint and the proximal tibia were exposed by a 6 cm anterior incision, and the joint was opened by a 3 cm transvastus approach, which allowed the patella to be dislocated laterally gaining full access to the patellar groove and femoral condyles. An osteochondral transverse defect, measuring 5 mm proximal to distal and spanning the entire width of the patellar groove was created using a jewelry saw and a chisel. In contrast to the original contour of the patellar groove, the base of the defect was flat and was an average of 1.5 to 2 mm below the surface of the middle of the patellar groove. The depth average at the medial and lateral edges was 2.5 to 4 mm. A drill was used to make a hole in every corner of the base of the defect and in the lateral and medial cortices adjacent to the defect. The osteochondral defect was located in a specific area of the patellar groove in which as the patella moves back and forth during the flexion and extension it would articulate with the periosteal graft. Using the same skin approach, after incising and retracting the deep fascia overlaying the medial aspect of the proximal tibia, a rectangular graft of the periosteum corresponding to the size of the osteochondral defect was harvested. The periosteal graft was inverted, and placed in the base of the defect with the cambium layer was facing up into the joint.

After surgery rabbits were allowed unrestricted motion for 6 weeks. The rabbits from the 6-week post-op group were sacrificed, and the specimens were harvested and analyzed as follows. The repair sites and corresponding contralateral sites were cut in half longitudinally. Half of the samples were used for biomechanical testing followed by GAG and collagen typing analyses, while the other half from each limb was processed for histology. The medial and lateral sides were evenly distributed between the outcome analyses to avoid potential bias.

The TGF-β1-injected periosteum was obviously thicker than the non-injected periosteum in the rabbits used in this surgical study. The TGF-β1-injected periosteum was also easier to elevate from the bone, and it did not shrink noticeably after elevation, as does untreated periosteum. In addition, the TGF-β1-injected periosteum was firmer, while remaining flexible, and was generally easier to handle during surgery.

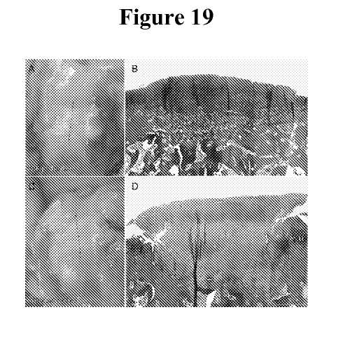

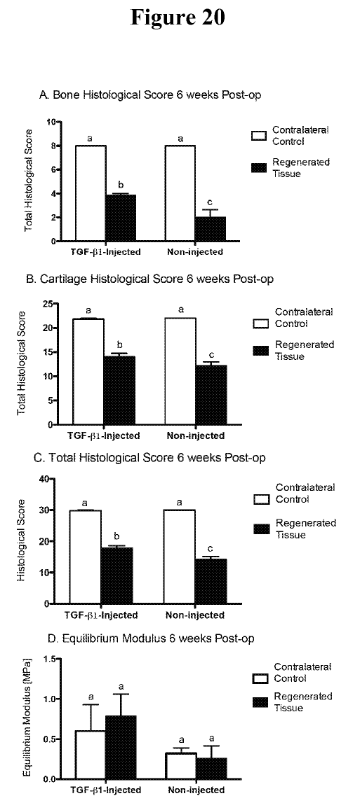

Results from a portion of the 6-week post-op group are summarized as follows. Gross analysis demonstrates complete filling of the defects with regenerated tissue in both the TGF-β1-injected and control groups with integration into the surrounding tissue and reformation of the original contours of the patellar groove (FIG. 19). The histological specimens (11 of 19 specimens) were scored by a blinded observer according to the nature of the predominant tissue, affinity of its matrix for the safranin O stain, surface regularity, structural integrity, bonding to the adjacent articular cartilage, and the surface level of the newly formed tissue compared with that of the adjacent cartilage as described herein (Table 8). The score was divided into separate bone (FIG. 20A) and cartilage (FIG. 20B) scores, and summed for a total histological score (FIG. 20C). The total score for the regenerated bone tissue in the TGF-β1-injected group (3.8±0.4, n=6) was significantly higher (p=0.0002) than the non-injected control group (2.0±1.4, n=5) (FIG. 20A). This observation is also apparent in the histology samples (FIG. 19). All of the specimens from the TGF-β1-injected group (n=6) exhibited partial bone infiltration into the defect area and integration with the surrounding tissue compared to only 40% of the control samples. Also, while greater than 80% of the specimens from the TGF-β1-injected group exhibited a visible tidemark, only 20% of the samples from the control group exhibited a tidemark present.

TABLE 8
Scoring system.

| | | | |
|---|---|---|---|
| Scoring criteria for cartilage | Neocartilage | 6 | Articular cartilage (AC)-like |
| layer reconstruction | morphology | 4 | AC-like > undifferentiated tissue |
| | | 2 | Undifferentiated tissue > AC-like |
| | | 0 | Undifferentiated tissue |
| | Surface regularity | 4 | Smooth and intact |
| | | 3 | Superficial surface disruption |
| | | 1 | Deep surface disruption |
| | | 0 | Severe surface disruption |
| | Neocartilage | 3 | Normal cellularity, no clusters |
| | Cellularity | 2 | Normal cellularity, with clusters |
| | (freedom from | 1 | Abnormal cellularity, no clusters |
| | degeneration) | 0 | Abnormal cellularity, with clusters |
| | Structural Integrity | 2 | No disruption |
| | | 1 | Slight disruption |
| | | 0 | Severe disruption |
| | Neocartilage | 2 | Level |
| | alignment with | 1 | Elevated |
| | surrounding AC | 0 | Depressed |
| | Integration | 3 | Integrated |
| | | 1 | Partially integrated |
| | | 0 | Not integrated |
| | Degeneration of | 2 | No degeneration |
| | adjacent AC | 1 | Mild degeneration |
| | | 0 | Severe degeneration |
| Scoring criteria for bone layer | Subchondral bone | 2 | Level |
| reconstruction | alignment | 1 | Depressed |
| | | 0 | Elevated |
| | Bone integration | 2 | Integrated |
| | | 1 | Partially Integrated |
| | | 0 | Not Integrated |
| | Bone infiltration into | 2 | Nearly complete or complete |
| | defect area | 1 | Partial |
| | | 0 | None or minimal |
| | Tidemark continuity | 2 | Present, no gaps |
| | | 1 | Present, with gaps |
| | | 0 | Absent |

Neocartilage formation was observed in both groups (FIG. 19). The cartilage score (FIG. 20B) in the TGF-β1-injected group (14±1.8, n=6) was also significantly higher (p=0.024) than the non-injected control group (12.2±1.8, n=5). Integration of the neocartilage was better in the TGF-β1-injected group with all of the specimens at least partially integrated, whereas 80% of the control samples were not integrated with the surrounding cartilage. Also, while surface regularity in half of the samples in the TGF-β1-injected group were scored as smooth and intact, none of the control samples received this score. Degeneration of the adjacent cartilage was not observed in any of the samples in either group. The total histological score (FIG. 20C) in the TGF-β31-injected group (17.8±1.8, n=6) was also significantly higher (p=0.0002) then in the control group (14.2±2.0, n=5). No significant difference in the equilibrium modulus was found between any of the groups (FIG. 20D). The histological scores in both of the tissue regeneration groups are significantly lower than the contralateral controls at 6 weeks post-op. Clearly, the tissue regeneration process is incomplete at 6 weeks as expected.

The results provided herein demonstrate that local injection of TGF-β1 alone or in combination with IGF-1 can be used to increase cambium layer thickness and in vitro and in vivo cartilage production in adult animals. The cambium cellularity, thickness, and cartilage formation values in the growth factor treated 6-24 month old rabbits in these studies are comparable to previous observations in younger rabbits, suggesting that periosteum in older rabbits can be rejuvenated for tissue engineering.

The results provided herein also demonstrate the feasibility of using TGF-β1-rejuvenated periosteum for the regeneration of osteochondral tissue in vivo. The TGF-β1-injected periosteum was easier to elevate from the bone, it was firmer and generally easier to handle during surgery. Therefore, it is possible that the use of rejuvenated periosteum will be less technically demanding and therefore, the clinical results may be more consistent compared to normal periosteal grafts.

The tissue regeneration process is incomplete at 6 weeks post-op. Therefore, it is predicted that the regenerated tissue from the rejuvenated periosteum group will more closely resemble the normal tissue (contralateral control) at both 6 & 12 months post-op, compared to 6 weeks post-op.

If this simple approach to periosteal rejuvenation is translated to the clinic, the number of patients who could benefit from the use of periosteum for tissue engineering or regeneration of cartilage could be greatly increased. In addition, the potential benefits of this approach also can extend to the use of periosteum for bone regeneration.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for obtaining a graft comprising periosteal tissue, wherein said method comprises:
(a) administering a composition to periosteal tissue of a mammal to form in vivo-treated periosteal tissue, wherein said composition comprises a growth factor, and
(b) harvesting said in vivo-treated periosteal tissue from said mammal, thereby obtaining said graft.

2. The method of claim 1, wherein said mammal is a human.

3. The method of claim 1, wherein said periosteal tissue is in an arm of said mammal.

4. The method of claim 1, wherein said periosteal tissue is in a leg of said mammal.

5. The method of claim 1, wherein said growth factor is a transforming growth factor or an insulin-like growth factor.

6. The method of claim 5, wherein said transforming growth factor is a transforming growth factor-beta polypeptide.

7. The method of claim 5, wherein said insulin-like growth factor is a insulin-like growth factor-1 polypeptide.

8. The method of claim 5, wherein said insulin-like growth factor is a insulin-like growth factor-2 polypeptide.

9. The method of claim 1, wherein said composition comprises a transforming growth factor and an insulin-like growth factor.

10. The method of claim 1, wherein said administration is a subperiosteal injection.

11. A method for regenerating cartilage or bone in a mammal, wherein said method comprises inserting a periosteal tissue graft into said mammal, wherein said periosteal tissue graft was harvested from said mammal after being treated in vivo with a composition comprising a growth factor.

12. The method of claim 11, wherein said mammal is a human.

13. The method of claim 11, wherein said periosteal tissue was harvested from an arm of said mammal.

14. The method of claim 11, wherein said periosteal tissue was harvested from a leg of said mammal.

15. The method of claim 11, wherein said growth factor is a transforming growth factor or an insulin-like growth factor.

16. The method of claim 15, wherein said transforming growth factor is a transforming growth factor-beta polypeptide.

17. The method of claim 15, wherein said insulin-like growth factor is a insulin-like growth factor-1 polypeptide.

18. The method of claim 15, wherein said insulin-like growth factor is a insulin-like growth factor-2 polypeptide.

19. The method of claim 11, wherein said composition comprises a transforming growth factor and an insulin-like growth factor.

20. The method of claim 11, wherein said periosteal tissue graft was treated via a subperiosteal injection of said composition.
